SEQUENCE LISTING (21 sequences)

SEQ ID NO: 1 - DNA, 363 bp, Mouse
gaattgaagcttgaggagtctggaggaggcttggtgcagcctggaggatccatgaaactctcttgtgctgcctctggattcacttttagtgacgcctggatggactgggtccgccagtctccagagaaggggcttgagtggattgctgaaattagaagcaaagctaataattatgcaacatactttgctgagtctgtgaaagggaggttcaccatctcaagagatgattccaaaagtggtgtctacctgcaaatgaacaacttaagagctgaggacactggcatttatttctgtaccaagttatcactacgttactggttcttcgatgtctggggcgcagggaccacggtcaccgtctcctca

SEQ ID NO: 2 - protein, 121 aa, Mouse
ELKLEESGGGLVQPGGSMKLSCAASGFTFSDAWMDWVRQSPEKGLEWIAEIRSKANNYATYFAESVKGRFTISRDDSKSGVYLQMNNLRAEDTGIYFCTKLSLRYWFFDVWGAGTTVTVSS

SEQ ID NO: 3 - DNA, 339 bp, Mouse
gattttgtgatgacccagactccactcactttgtcggttaccattggacaaccagcctccatctcttgcaagtcaagtcagagcctcttggatagtgatggaaagacatatttgaattggttgttacagaggccaggccagtctccaaagcacctcatctatctggtgtctaaactggactctggagtccctgacaggttcactggcagtggatcagggaccgatttcacactgagaatcagcagagtggaggctgaggatttgggagtttattattgctggcaaagtacacattttccgtggacgttcggtggaggcaccaagctggaaatcaaacgg

SEQ ID NO: 4 - protein, 113 aa, Mouse
DFVMTQTPLTLSVTIGQPASISCKSSQSLLDSDGKTYLNWLLQRPGQSPKHLIYLVSKLDSGVPDRFTGSGSGTDFTLRISRVEAEDLGVYYCWQSTHFPWTFGGGTKLEIKR

SEQ ID NO: 5 - DNA, 420 bp, Artificial Sequence
  Oligonucleotide
atgtacttgggactgaactatgttttcatcgtttttctcctgaatggtgtccagagtgaagtgcagcttgtggagtctggaggaggcttggtgcagcctggaggatccctgaaactctcttgtgctgcctctggattcacttttagtgacgcctggatggactgggtccgccaggcttccgggaaggggcttgagtggattgctgaaattagaagcaaagctaataattatgcaacatactttgctgagtctgtgaaagggaggttcaccatctcaagagatgattccaaaaacaccgcctacctgcaaatgaacagcttaaaaaccgaggacactgccgtttattactgtaccaagttatcactgcgttactggttcttcgatgtctggggccaggggaccctggtcaccgtctcctca

SEQ ID NO: 6 - protein, 140 aa, Artificial Sequence
  Polypeptide
MYLGLNYVFIVFLLNGVQSEVQLVESGGGLVQPGGSLKLSCAASGFTFSDAWMDWVRQASGKGLEWIAEIRSKANNYATYFAESVKGRFTISRDDSKNTAYLQMNSLKTEDTAVYYCTKLSLRYWFFDVWGQGTLVTVSS

SEQ ID NO: 7 - DNA, 363 bp, Artificial Sequence
  Oligonucleotide
gaagtgcagcttgtggagtctggaggaggcttggtgcagcctggaggatccctgaaactctcttgtgctgcctctggattcacttttagtgacgcctggatggactgggtccgccaggcttccgggaaggggcttgagtggattgctgaaattagaagcaaagctaataattatgcaacatactttgctgagtctgtgaaagggaggttcaccatctcaagagatgattccaaaaacaccgcctacctgcaaatgaacagcttaaaaaccgaggacactgccgtttattactgtaccaagttatcactgcgttactggttcttcgatgtctggggccaggggaccctggtcaccgtctcctca

SEQ ID NO: 8 - protein, 121 aa, Artificial Sequence
  Polypeptide
EVQLVESGGGLVQPGGSLKLSCAASGFTFSDAWMDWVRQASGKGLEWIAEIRSKANNYATYFAESVKGRFTISRDDSKNTAYLQMNSLKTEDTAVYYCTKLSLRYWFFDVWGQGTLVTVSS

SEQ ID NO: 9 - DNA, 396 bp, Artificial Sequence
  Oligonucleotide
atgatgagtcctgcccagttcctgtttctgttggtgctctggattcgggaaaccaacggtgatatcgtgatgacccagactccactctctttgtccgttacccctggacaaccagcctccatctcttgcaagtcaagtcagagcctcttggatagtgatggaaagacatatttgaattggttgttacagaagccaggccagtctccacagcacctcatctatctggtgtctaaactggactctggagtccctgacaggttcagtggcagtggatcagggaccgatttcacactgaaaatcagcagagtggaggctgaggatgttggagtttattattgctggcaaagtacacattttccctggaccttcggtggaggcaccaaggtggaaatcaaa

SEQ ID NO: 10 - protein, 132 aa, Artificial Sequence
  Polypeptide
MMSPAQFLFLLVLWIRETNGDIVMTQTPLSLSVTPGQPASISCKSSQSLLDSDGKTYLNWLLQKPGQSPQHLIYLVSKLDSGVPDRFSGSGSGTDFTLKISRVEAEDVGVYYCWQSTHFPWTFGGGTKVEIK

SEQ ID NO: 11 - DNA, 336 bp, Artificial Sequence
  Oligonucleotide
gatatcgtgatgacccagactccactctctttgtccgttacccctggacaaccagcctccatctcttgcaagtcaagtcagagcctcttggatagtgatggaaagacatatttgaattggttgttacagaagccaggccagtctccacagcacctcatctatctggtgtctaaactggactctggagtccctgacaggttcagtggcagtggatcagggaccgatttcacactgaaaatcagcagagtggaggctgaggatgttggagtttattattgctggcaaagtacacattttccctggaccttcggtggaggcaccaaggtggaaatcaaa

SEQ ID NO: 12 - protein, 112 aa, Artificial Sequence
  Polypeptide
DIVMTQTPLSLSVTPGQPASISCKSSQSLLDSDGKTYLNWLLQKPGQSPQHLIYLVSKLDSGVPDRFSGSGSGTDFTLKISRVEAEDVGVYYCWQSTHFPWTFGGGTKVEIK

SEQ ID NO: 13 - protein, 112 aa, Artificial Sequence
  Polypeptide
DFVMTQTPLSLSVTPGQPASISCKSSQSLLDSDGKTYLNWLLQKPGQSPQHLIYLVSKLDSGVPDRFSGSGSGTDFTLKISRVEAEDVGVYYCWQSTHFPWTFGGGTKVEIK

SEQ ID NO: 14 - protein, 457 aa, Mouse
MRVLILLWLFTAFPGILSVVQLQESGPGLVKPSQSLSLTCTVTGYSITSDYAWNWIRQFPGNRLEWMGYIDYSGSTKYNPSLKSRISVTRDTSKNQFFLQLNSVTTEDTATYYCARDFGDAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 15 - DNA, 1374 bp, Mouse
atgagagtgctgattcttttgtggctgttcacagcctttcctggtatcctgtctgttgtgcagcttcaggagtcgggacctggcctggtgaaaccttctcagtctctgtccctcacctgcactgtcactggctactcaatcaccagtgattatgcctggaactggatccggcagtttccaggaaacagactggagtggatgggctacatagactacagtggtagcactaaatacaacccctctctcaaaagtcgaatctctgtcactcgagacacatccaagaaccagttcttcctgcagttgaattctgtgactactgaggacacagccacatattactgtgcaagagactttggtgatgcttactggggccaggggactctggtcactgtctctgcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcaaacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcaaatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 16 - protein, 240 aa, Mouse
MDSQAQVLMLLLLWVSGSCGDIVMSQSPSSLAVSVGEKVTMSCKSSQSLLYSRNQKNYLAWYQLKPGQSPKLLIYWASTRESGVPDRFTGSGSGTDFTLTISSVKAEDLAVYYCQQYYSYPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 17 - DNA, 723 bp, Mouse
atggattcacaggcccaggttcttatgttactgctgctatgggtatctggttcctgtggggacattgtgatgtcacagtctccatcctccctagctgtgtcagttggagagaaggttactatgagctgcaagtccagtcagagccttttatatagtaggaatcaaaagaactacttggcctggtaccagctgaagccagggcagtctcctaaactgctgatttactgggcatccactagggaatctggggtccctgatcgcttcacaggcagtggatctgggacagatttcactctcaccatcagcagtgtgaaggctgaagacctggcagtttattactgtcagcaatattatagctatccgctcacgttcggtgctgggaccaagctggagctgaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 18 - protein, 460 aa, Mouse
MNFGLSLIFLALILKGVQCEVQLVEAGGDLVKPGGSLKLSCAASGFSLSNYGMSWVRQTPDKRLEWVASISSGGRFTSYPDSVRGRFTISRDNAKNTLYLQMSGLKSEDTAMYYCARHDGNGGDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 19 - DNA, 1383 bp, Mouse
atgaacttcgggctcagcctgattttccttgccctcattttaaaaggtgtccagtgtgaggtgcagctggtggaggctgggggagacttagtgaagcctggagggtccctgaaactctcctgtgcggcctctggattcagtttgagtaactatggcatgtcctgggttcgccagactccagacaagaggctggagtgggtcgcaagcattagtagtggtggtcgtttcacctcctatccagacagtgtgagggggcgattcaccatctccagagacaatgccaagaacaccctgtacctgcaaatgagcggtctgaagtctgaggacacagccatgtattactgtgcaagacacgacggcaacggtggggactactggggtcaaggaacctcagtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 20 - protein, 235 aa, Mouse
MDFQVQIFSFLLISASVIMSRGQIVLSQFPAILSASPGEKVTMTCRARSSVSFMHWYQQKPGSSPKPWIYATSNLASGVPPRFSGSGSGTSYSLTISRVEAEDAATYYCQQWSSNPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 21 - DNA, 708 bp, Mouse
atggattttcaagtgcagattttcagcttcctgctaatcagtgcttcagtcataatgtccagaggacaaattgttctctcccagtttccagcaatcctgtctgcatctccaggggagaaggtcacaatgacttgcagggccaggtcaagtgtaagtttcatgcactggtaccagcagaagccaggatcctcccccaaaccctggatttatgccacatccaacctggcttctggagtccctcctcgcttcagtggcagtgggtctgggacctcttactctctcacaatcagcagagtggaggctgaagatgctgccacttattactgccagcagtggagtagtaacccatacacgttcggaggggggactaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

(19) United States
(12) Patent Application Publication
(10) Pub. No.: US 2017/0348389 A1
(43) Pub. Date: Dec. 7, 2017

(54) TREATING LIVER DISEASES WITH INTERLEUKIN 24

(71) Applicant: National Cheng Kung University, Tainan City (TW)

(72) Inventor: Ming-Shi Chang, Tainan City (TW)

(73) Assignee: National Cheng Kung University, Tainan City (TW)

(21) Appl. No.: 15/173,570

(22) Filed: Jun. 3, 2016

(51) Int. Cl. A61K 38/20; A61K 39/395; A61K 39/00

(52) U.S. Cl. CPC A61K 38/20; A61K 39/3955; A61K 2039/505

(57) ABSTRACT

Methods of treating liver diseases such as liver fibrosis using an interleukin 24 (IL-24) protein.

### BACKGROUND OF THE INVENTION

Liver disease such as hepatic steatosis and liver fibrosis are important causes of morbidity and mortality worldwide. Chronic hepatocellular damage results in the development of liver fibrosis and subsequently liver cirrhosis. Liver fibrosis is the scarring process, in which extracellular matrix proteins, including collagens, accumulate in the liver for damage repair. Liver cirrhosis involves regeneration of nodules surrounded by fibrous bands. It is an advanced stage of liver fibrosis accompanied with distortion of the hepatic vasculature. Liver steatosis, also known as fatty liver diseases, involves accumulation of fat in the liver. Fatty liver diseases include alcohol-related fatty liver disease and non-alcohol fatty liver disease.

Interleukin 24 (IL-24), also known as melanoma differentiation-associated 7 (mda-7), is a cytokine belonging to the IL-10 family. IL-24 is predominantly produced by immune cells such as activated monocytes, macrophages, and T cells. It has been reported that IL-24 may play roles in controlling cell survival and proliferation by inducing rapid activation of transcription factors STAT1 and STAT3.

### SUMMARY OF THE INVENTION

The present disclosure is based on the unexpected results that IL-24 successfully reduced symptoms of liver diseases such as liver fibrosis and prolonged survival rate of mice suffering from liver injuries.

Accordingly, one aspect of the present disclosure features a method for alleviating or delaying the onset of a liver disease (e.g., liver fibrosis, liver cirrhosis, or liver steatosis), the method comprising administering to a subject in need thereof an effective amount of a pharmaceutical composition comprising an interleukin 24 (IL-24) protein, such as a human IL-24.

In some embodiments, the subject is a human patient having or suspected of having the liver disease for example, liver fibrosis, which may be associated with chronic HBV infection, chronic HCV infection, alcohol abuse, nonalcoholic steatohepatitis, autoimmune hepatitis, primary biliary cirrhosis, fatty liver disease, liver cancer, or an idiopathic liver disease.

In any of the methods described herein, the IL-24 protein can be administered by a systemic route. In some examples, the IL-24 protein is administered parenterally. For example, the IL-24 protein can be administered via intravenous infusion or intraperitoneal injection.

Any of the methods described herein may further comprise administering to the subject an effective amount of a pharmaceutical composition comprising an interleukin 20 (IL-20) antagonist, which may be an antibody binding to human IL-20 or an antibody binding to a human IL-20 receptor, e.g., those described herein. In some examples, the antibody binds to subunit R1 of a human IL-20 receptor. Any of the antibodies used in the methods described herein can be a full-length antibody or an antigen-binding fragment thereof. In some instances, the antibody can be a human antibody or a humanized antibody.

Also within the scope of the present disclosure are pharmaceutical compositions comprising any of the IL-24 proteins disclosed herein for use in treating a liver disease such as liver fibrosis/cirrhosis or liver steatosis; and uses of any of the IL-24 proteins in manufacturing a medicament for use in treating the target liver disease.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

FIG. 1 includes diagrams showing the protective effects of IL-24 in C57BL/6Jnarl mice treated with TAA. (A) Survival rate were observed at 0, 24, 48 and 72 h after TAA treatment. (B) Body weight loss was measured at 48 h after TAA treatment. (C) H/E staining of liver sections from mice were used to assess the necrosis area at 72 h after TAA treatment. (D) Serum AST and ALT were measured at 24 and 72 h after TAA treatment. Data are means±SEM. *P<0.05 versus TAA.

FIG. 2 is a diagram showing the reduction of fibrosis and inflammatory markers in mice treated with both TAA and IL-24. The mRNA expression of TGF-β, α-SMA, MIP-2β, TIMP-1, MCP-1, IL-1β, KC and TNF-α in liver of mice was analyzed using qRT-PCR at 72 h after treatment. Data are means±SEM. *P<0.05 versus TAA.

### DETAILED DESCRIPTION OF THE INVENTION

The present disclosure is based on the unexpected results that interleukin 24 (IL-24) showed protective effects in a well-recognized mouse model for human liver diseases. Specifically, IL-24 was found to protect mice from short-term TAA-induced liver injury, including increased survival rate of the mice having liver injuries, alleviated body weight loss from liver injuries, decreased serum levels of ALT and AST in the mice with liver injuries, and reduced the fibrosis markers (e.g., TGF-β, α-SMA, MIP-2β, TIMP-1) and inflammation molecules (e.g., MCP-1, IL-1β, KC and TNF-α) in the liver of mice treated with IL-24 after TAA-induced fibrosis.

Accordingly, provided herein are methods of alleviating and/or delaying the onset of a liver disease with an effective amount of an IL-24 protein.

### I. IL-24 Proteins

IL-24 is a member of the IL-10 cytokine superfamily. The structural and functional information of this cytokine were well known in the art. IL-24 binds two heterodimeric receptors, IL-20R1/IL-20R2 and IL-22R1/IL-20R2 and activates rapidly transcription factors Stat-1 and Stat-3, which play essential roles in cell survival and proliferation. Wang et al., Immunology, 114:166-170 (2005). IL-24 proteins are highly homologous across species. The human IL-24 shares close to 70% sequence identity to mouse and rat IL-24, indicating that this cytokine may function across species. Rodent IL-24 proteins were found to be able to activate human IL-24 receptors. Wang et al., Genes Immun 5:363-370 (2004).

Mature human IL-24 is a glycoprotein having a molecular weight of around 33,000 kDal. The amino acid sequence of an exemplary human IL-24 can be found under GenBank accession no. AAH09681.1. Mature rat and mouse IL-24 have a molecular weight of around 23,000 kDal. Amino acid sequences of exemplary rat and mouse IL-24 can be found under GenBank accession no. NP_579845.1 and NP_444325.2.

The IL-24 proteins described herein refer to polypeptides having the same bioactivity as a naturally-occurring IL-24, for example, the human IL-24. In some embodiments, the IL-24 proteins used in the method described herein is a native IL-24 protein obtained from a suitable species, e.g., human, a non-human primate such as monkey, rat, mouse, pig, bovine, rabbit, etc. Such native IL-24 proteins include naturally-occurring isoforms such as polymorphism variants and slice variants.

In some examples, the IL-24 protein is a human protein. Examples include the IL-24 protein described in GenBank accession no. AAH09681.1, human IL-24 isoform 1 described in GenBank accession no. NP_006841.1, human IL-24 isoform 3 described in GenBank accession no. NP_001172085.1, human IL-24 isoform 4 described in GenBank accession no. NP_001172086.1, human IL-24 isoform 5 described in GenBank accession no. NP_001172087.1, human IL-24 splice variant delE3 described in GenBank accession no. AAV52800.1, and human IL-24 splice variant delE5 described in GenBank accession no. AAV52801.1.

In other examples, the IL-24 protein is a native IL-24 protein derived from a suitable mammal such as a non-human primate, rat, mouse, pig, bovine, rabbit, etc. Examples include gorilla IL-24 (e.g., that disclosed under GenBank accession no. XP_004028343), a chimpanzee IL-24 (e.g., that disclosed under GenBank accession no. XP_008974768.1), orangutan IL-24 (e.g., those disclosed under GenBank accession nos. XP_002809582.1 and XP_009236749.1), a bovine IL-24 (such as that described under GenBank accession nos. XP_010363962), and a rodent IL-24 such as the rat and mouse IL-24 proteins described above.

The IL-24 protein described herein may be a native IL-24 protein that shares at least about 65% (e.g., about 70%, 75%, 80%, 85%, 90%, 95%, 98%, or above) sequence identity to a human counterpart as those described herein. The term “about” allows for an up to 5% variation from the reference value. The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

Alternatively, the IL-24 protein can be a functional variant of a naturally-occurring IL-24 protein. A functional variant of a naturally-occurring IL-24 can share a high sequence homology with the native counterpart (e.g., at least about 80%, 85%, 90%, 95%, 98% or above) and contain one or more mutations relative to the native counterpart. Typically, such mutations should occur at regions that do not substantially affect the bioactivity of the IL-24 protein. Structural features of IL-24, as a member of the IL-19 subfamily, were well known in the art. Zdanov, Vitam Horm, 74:61-76 (2006). Functional domains of IL-24 can also be identified by comparing the amino acid sequence of a particular IL-24 protein with other members of the IL-24 family and/or the IL-10/IL-19 family. In some instances, the functional variant contains up to 10 (e.g., 9, 8, 7, 6, 5, 4, 3, 2, or 1) amino acid substitutions as compared with the native counterpart. The activity of a candidate functional variant can be verified by routine methodology.

The skilled artisan will realize that conservative amino acid substitutions may be made to a wild-type IL-24 to provide functional variants, i.e., the variants retain the functional capabilities of the particular IL-24 protein. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

Conservative amino-acid substitutions in the amino acid sequence of a native IL-24 to produce functional variants typically are made by alteration of a nucleic acid encoding the native IL-24. Such substitutions can be made by a variety of methods known to one of ordinary skill in the art. For example, amino acid substitutions may be made by PCR-directed mutation, site-directed mutagenesis according to the method of Kunkel (Kunkel, PNAS 82: 488-492, 1985), or by chemical synthesis of a nucleic acid molecule encoding an IL-24 protein.

Any of the IL-24 proteins described herein can be prepared by routine technology, for example, recombinant technology. Native IL-24 proteins can also be isolated from a suitable natural source.

### II. Pharmaceutical Compositions

An IL-24 protein as described herein can be mixed with a pharmaceutically acceptable carrier (excipient), including buffer, to form a pharmaceutical composition for use in alleviating a liver disease such as liver fibrosis/cirrhosis or liver steatosis. “Acceptable” means that the carrier must be compatible with the active ingredient of the composition (and preferably, capable of stabilizing the active ingredient) and not deleterious to the subject to be treated. Pharmaceutically acceptable excipients (carriers) including buffers, which are well known in the art. See, e.g., Remington: The Science and Practice of Pharmacy 20th Ed. (2000) Lippincott Williams and Wilkins, Ed. K. E. Hoover.

The pharmaceutical compositions to be used in the present methods can comprise pharmaceutically acceptable carriers, excipients, or stabilizers in the form of lyophilized formulations or aqueous solutions. (Remington: The Science and Practice of Pharmacy 20th Ed. (2000) Lippincott Williams and Wilkins, Ed. K. E. Hoover). Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Pharmaceutically acceptable excipients are further described herein.

In some examples, the pharmaceutical composition described herein comprises liposomes containing the IL-24 protein, which can be prepared by methods known in the art, such as described in Epstein, et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The active ingredients (e.g., an IL-24 protein) may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are known in the art, see, e.g., Remington, The Science and Practice of Pharmacy 20th Ed. Mack Publishing (2000).

In other examples, the pharmaceutical composition described herein can be formulated in sustained-release format. Suitable examples of sustained-release preparations include semipermeable matrices of solid hydrophobic polymers containing the IL-24 protein, which matrices are in the form of shaped articles, e.g. films, or microcapsules. Examples of sustained-release matrices include polyesters, hydrogels (for example, poly(2-hydroxyethyl-methacrylate), or poly(v nylalcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The pharmaceutical compositions to be used for in vivo administration must be sterile. This is readily accomplished by, for example, filtration through sterile filtration membranes. Therapeutic IL-24-containing compositions are generally placed into a container having a sterile access port, for example, an intravenous solution bag or vial having a stopper pierceable by a hypodermic injection needle.

The pharmaceutical compositions described herein can be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Suitable surface-active agents include, in particular, non-ionic agents, such as polyoxyethylenesorbitans (e.g., Tween™ 20, 40, 60, 80 or 85) and other sorbitans (e.g., Span™ 20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

Suitable emulsions may be prepared using commercially available fat emulsions, such as Intralipid™, Liposyn™, Infonutrol™, Lipofundin™, and Lipiphysan™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion can comprise fat droplets between 0.1 and 1.0 μm, particularly 0.1 and 0.5 μm, and have a pH in the range of 5.5 to 8.0.

The emulsion compositions can be those prepared by mixing an IL-24 protein with Intralipid™ or the components thereof (soybean oil, egg phospholipids, glycerol and water, etc.).

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect.

Compositions in preferably sterile pharmaceutically acceptable solvents may be nebulised by use of gases. Nebulised solutions may be breathed directly from the nebulising device or the nebulising device may be attached to a face mask, tent or intermittent positive pressure breathing machine. Solution, suspension or powder compositions may be administered, preferably orally or nasally, from devices which deliver the formulation in an appropriate manner.

### III. Treating Liver Diseases

To practice the method disclosed herein, an effective amount of the pharmaceutical composition described above can be administered to a subject (e.g., a human) in need of the treatment via a suitable route, such as intravenous administration, e.g., as a bolus or by continuous infusion over a period of time, by intramuscular, intraperitoneal, intracerebrospinal, subcutaneous, intra-articular, intrasynovial, intrathecal, oral, inhalation or topical routes. Commercially available nebulizers for liquid formulations, including jet nebulizers and ultrasonic nebulizers are useful for administration. Liquid formulations can be directly nebulized and lyophilized powder can be nebulized after reconstitution. Alternatively, an IL-24 protein can be aerosolized using a fluorocarbon formulation and a metered dose inhaler, or inhaled as a lyophilized and milled powder.

The subject to be treated by the methods described herein can be a mammal, more preferably a human. Mammals include, but are not limited to, farm animals, sport animals, pets, primates, horses, dogs, cats, mice and rats. A human subject who needs the treatment may be a human patient having, at risk for, or suspected of having a liver disease, including liver fibrosis (e.g., liver cirrhosis) and liver steatosis (also known as fatty liver disease), which may be alcohol-associated or non-alcohol associated. A subject having a liver disease, e.g., those described herein, can be identified by routine medical examination, e.g., laboratory tests, liver functions tests, liver biopsy, CT scans, or ultrasounds. A subject suspected of having a liver disease such as liver fibrosis might show one or more symptoms of the disorder, e.g., elevated levels of aminotransferases (AST and ALT), levels of alkaline phosphatase and gamma-glutamyl transferase, elevated levels of bilirubin (a marker for cirrhosis progresses), decreased level of albumin, increased prothrombin time, elevated levels of globulin, leukopenia and neutropenia, and/or coagulation defects. A subject at risk for the liver disease can be a subject having one or more of the risk factors for that disorder. For example, risk factors associated with liver fibrosis and/or liver steatosis include (a) viral infection, particularly HBV or HCV infection, (b) age (liver fibrosis is more frequent in people over 50), (c) gender (occur more rapidly in men than in women), (d) heavy alcohol consumption, (e) fatty liver, and (f) insulin resistance.

“An effective amount” as used herein refers to the amount of each active agent required to confer therapeutic effect on the subject, either alone or in combination with one or more other active agents. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

Empirical considerations, such as the half-life, generally will contribute to the determination of the dosage. For example, human-based IL-24 proteins may be used to prolong half-life of the cytokine and to prevent the cytokine being attacked by the host's immune system. Frequency of administration may be determined and adjusted over the course of therapy, and is generally, but not necessarily, based on treatment and/or suppression and/or amelioration and/or delay of the liver disease (e.g., liver fibrosis/cirrhosis or liver steatosis). Alternatively, sustained continuous release formulations of an IL-24 protein may be appropriate. Various formulations and devices for achieving sustained release are known in the art.

In one example, dosages for an IL-24 protein as described herein may be determined empirically in individuals who have been given one or more administration(s) of the IL-24 protein. Individuals are given incremental dosages of the cytoline. To assess efficacy of the cytokine, an indicator of the live disease such as liver fibrosis (e.g., levels of AST and ALT) can be followed.

Generally, for administration of any of the IL-24 protein described herein, an initial candidate dosage can be about 0.5-2 mg/kg. For the purpose of the present disclosure, a typical daily dosage might range from about any of 0.1 μg/kg to 3 μg/kg to 30 μg/kg to 300 μg/kg to 3 mg/kg, to 30 mg/kg to 100 mg/kg or more, depending on the factors mentioned above. For repeated administrations over several days or longer, depending on the condition, the treatment is sustained until a desired suppression of symptoms occurs or until sufficient therapeutic levels are achieved to alleviate liver fibrosis or cirrhosis, or a symptom thereof. An exemplary dosing regimen comprises administering an initial dose of about 2 mg/kg, followed by a weekly maintenance dose of about 1 mg/kg of the IL-24 protein, or followed by a maintenance dose of about 1 mg/kg every other week. However, other dosage regimens may be useful, depending on the pattern of pharmacokinetic decay that the practitioner wishes to achieve. For example, dosing from one-four times a week is contemplated. In some embodiments, dosing ranging from about 3 μg/mg to about 2 mg/kg (such as about 3 μg/mg, about 10 μg/mg, about 30 μg/mg, about 100 μg/mg, about 300 μg/mg, about 1 mg/kg, and about 2 mg/kg) may be used. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays. The dosing regimen (including the IL-24 protein used) can vary over time. The particular dosage regimen, i.e., dose, timing and repetition, will depend on the particular individual and that individual's medical history, as well as the properties of the individual agents (such as the half-life of the agent, and other considerations well known in the art).

For the purpose of the present disclosure, the appropriate dosage of an IL-24 protein will depend on the specific IL-24 protein employed, the type and severity of liver disease, whether the IL-24 is administered for preventive or therapeutic purposes, previous therapy, the patient's clinical history and response to the antagonist, and the discretion of the attending physician. Typically the clinician will administer an IL-24 protein, such as a human IL-24 protein, until a dosage is reached that achieves the desired result. Administration of an IL-24 protein can be continuous or intermittent, depending, for example, upon the recipient's physiological condition, whether the purpose of the administration is therapeutic or prophylactic, and other factors known to skilled practitioners. The administration of an IL-24 protein may be essentially continuous over a preselected period of time or may be in a series of spaced dose, e.g., either before, during, or after developing liver fibrosis or cirrhosis.

As used herein, the term “treating” refers to the application or administration of a composition including one or more active agents to a subject, who has a liver disease such as liver fibrosis or liver steatosis, a symptom of the liver disease, or a predisposition toward the disease, with the purpose to cure, heal, alleviate, relieve, alter, remedy, ameliorate, improve, or affect the disorder, the symptom of the disease, or the predisposition toward the disease.

Alleviating the liver disease such as liver fibrosis/cirrhosis or liver steatosis includes delaying the development or progression of the disease, or reducing disease severity. Alleviating the disease does not necessarily require curative results. As used therein, “delaying” the development of a disease (such as liver fibrosis or cirrhosis) means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of liver fibrosis includes initial onset and/or recurrence.

In some embodiments, the IL-24 protein described herein is administered to a subject in need of the treatment at an amount sufficient to reduce the level of the enzymatic activity of AST and/ALT in the subject by at least about 20% (e.g., 30%, 40%, 50%, 60%, 70%, 80%, 90% or greater). In other embodiments, the amount of the IL-24 protein used in the treatment is sufficient to prolong the survival rate of the patient, particularly those having severe liver cirrhosis. Alternatively or in addition, the amount of the IL-24 protein is sufficient to reduce one or more fibrosis and/or inflammatory factors (e.g., TGF-β, α-SMA, MIP-2β, TIMP-1, MCP-1, IL-1β, KC and TNF-α) by at least about 20% (e.g., 30%, 40%, 50%, 60%, 70%, 80%, 90% or greater).

Conventional methods, known to those of ordinary skill in the art of medicine, can be used to administer the pharmaceutical composition to the subject, depending upon the type of disease to be treated or the site of the disease. This composition can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, intraperitoneal, and intracranial injection or infusion techniques. In addition, it can be administered to the subject via injectable depot routes of administration such as using 1-, 3-, or 6-month depot injectable or biodegradable materials and methods.

Injectable compositions may contain various carriers such as vegetable oils, dimethylacetamide, dimethylformamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the IL-24 protein and a physiologically acceptable excipients is infused. Physiologically acceptable excipients may include, for example, 5% dextrose, 0.9% saline, Ringer's solution or other suitable excipients. Intramuscular preparations, e.g., a sterile formulation of a suitable soluble salt form of the IL-24 protein, can be dissolved and administered in a pharmaceutical excipient such as Water-for-Injection, 0.9% saline, or 5% glucose solution.

In one embodiment, an IL-24 protein is administered via site-specific or targeted local delivery techniques. Examples of site-specific or targeted local delivery techniques include various implantable depot sources of the IL-24 protein or local delivery catheters, such as infusion catheters, an indwelling catheter, or a needle catheter, synthetic grafts, adventitial wraps, shunts and stents or other implantable devices, site specific carriers, direct injection, or direct application. See, e.g., PCT Publication No. WO 00/53211 and U.S. Pat. No. 5,981,568.

The therapeutic IL-24 proteins described herein can be delivered using gene delivery vehicles. The gene delivery vehicle can be of viral or non-viral origin (see generally, Jolly, Cancer Gene Therapy (1994) 1:51; Kimura, Human Gene Therapy (1994) 5:845; Connelly, Human Gene Therapy (1995) 1:185; and Kaplitt, Nature Genetics (1994) 6:148). Expression of such coding sequences can be induced using endogenous mammalian or heterologous promoters and/or enhancers. Expression of the coding sequence can be either constitutive or regulated.

Viral-based vectors for delivery of a desired polynucleotide and expression in a desired cell are well known in the art. Exemplary viral-based vehicles include, but are not limited to, recombinant retroviruses (see, e.g., PCT Publication Nos. WO 90/07936; WO 94/03622; WO 93/25698; WO 93/25234; WO 93/11230; WO 93/10218; WO 91/02805; U.S. Pat. Nos. 5,219,740 and 4,777,127; GB Patent No. 2,200,651; and EP Patent No. 0 345 242), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532)), and adeno-associated virus (AAV) vectors (see, e.g., PCT Publication Nos. WO 94/12649, WO 93/03769; WO 93/19191; WO 94/28938; WO 95/11984 and WO 95/00655). Administration of DNA linked to killed adenovirus as described in Curiel, Hum. Gene Ther. (1992) 3:147 can also be employed.

Non-viral delivery vehicles and methods can also be employed, including, but not limited to, polycationic condensed DNA linked or unlinked to killed adenovirus alone (see, e.g., Curiel, Hum. Gene Ther. (1992) 3:147); ligand-linked DNA (see, e.g., Wu, J. Biol. Chem. (1989) 264:16985); eukaryotic cell delivery vehicles cells (see, e.g., U.S. Pat. No. 5,814,482; PCT Publication Nos. WO 95/07994; WO 96/17072; WO 95/30763; and WO 97/42338) and nucleic charge neutralization or fusion with cell membranes. Naked DNA can also be employed. Exemplary naked DNA introduction methods are described in PCT Publication No. WO 90/11092 and U.S. Pat. No. 5,580,859. Liposomes that can act as gene delivery vehicles are described in U.S. Pat. No. 5,422,120; PCT Publication Nos. WO 95/13796; WO 94/23697; WO 91/14445; and EP Patent No. 0524968. Additional approaches are described in Philip, Mol. Cell. Biol. (1994) 14:2411, and in Woffendin, Proc. Natl. Acad. Sci. (1994) 91:1581.

It is also apparent that an expression vector can be used to direct expression of any of the IL-24 proteins. The particular dosage regimen, i.e., dose, timing and repetition, used in the method described herein will depend on the particular subject and that subject's medical history.

Treatment efficacy can be assessed by methods well-known in the art, e.g., monitoring the levels of AST and/or ALT in a patient subjected to the treatment. See, e.g., Example 1 below. See also U.S. Pat. No. 8,603,470.

### IV. Kits

The present disclosure also provides kits for use in alleviating a liver disease such as liver fibrosis/cirrhosis or liver steatosis. Such kits can include one or more containers comprising an IL-24 protein (such as those described herein). In some embodiments, the IL-24 protein is a human protein.

In some embodiments, the kit can comprise instructions for use in accordance with any of the methods described herein. The included instructions can comprise a description of administration of the IL-24 protein to treat, delay the onset, or alleviate the liver disease according to any of the methods described herein. The kit may further comprise a description of selecting an individual suitable for treatment based on identifying whether that individual has a target liver disease such as liver fibrosis or liver steatosis. In still other embodiments, the instructions comprise a description of administering an IL-24 protein to an individual at risk of the liver disease.

The instructions relating to the use of an IL-24 protein generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, delaying the onset and/or alleviating liver fibrosis or cirrhosis. Instructions may be provided for practicing any of the methods described herein.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is an IL-24 protein.

Kits may optionally provide additional components such as buffers and interpretive information. Normally, the kit comprises a container and a label or package insert(s) on or associated with the container. In some embodiments, the invention provides articles of manufacture comprising contents of the kits described above.

### V. Combined Therapy

Any of the IL-24 proteins described herein can be used in combination with one or more additional pharmaceutical agents (e.g., therapeutically and/or prophylactically active agents) useful in treating and/or delaying the onset of a liver disease, including those described herein, e.g., liver fibrosis/cirrhosis or liver steatosis. The IL-24 protein or compositions containing such can be administered in combination with the additional pharmaceutical agents that improve their activity (e.g., activity, including potency and/or efficacy) in treating and/or reducing the risk for a target liver disease in a subject in need thereof, improve bioavailability, improve safety, reduce drug resistance, reduce and/or modify metabolism, inhibit excretion, and/or modify distribution in a subject, biological sample, tissue, or cell. It will also be appreciated that the therapy employed may achieve a desired effect for the same disorder, and/or it may achieve different effects. In certain embodiments, a pharmaceutical composition described herein including an IL-24 protein as described herein and an additional pharmaceutical agent shows a synergistic effect that is absent in a pharmaceutical composition including one of the IL-24 protein and the additional pharmaceutical agent, but not both.

An IL-24 protein or a combination containing such can be administered concurrently with, prior to, or subsequent to the one or more additional pharmaceutical agents (e.g., therapeutically active agents or prophylactically active agents), which may be useful as, e.g., combination therapies in treating and/or reducing the risk for a liver disease such as those described herein. Pharmaceutical agents include small organic molecules such as drug compounds or co-crystals thereof (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, antibodies, small molecules linked to proteins such as antibodies, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful in treating and/or reducing the risk for a neuropsychiatric or glucose or lipid metabolic disorder in a subject. In certain embodiments, the additional pharmaceutical agent is a pharmaceutical agent approved by a regulatory agency (e.g., the US FDA) for treating and/or reducing the risk for a liver disease as described herein in a subject. Each additional pharmaceutical agent may be administered at a dose and/or on a time schedule determined for that pharmaceutical agent. The additional pharmaceutical agents may also be administered together with each other and/or with the IL-24 protein or a composition containing such in a single dose or administered separately in different doses. The particular combination to employ in a regimen will take into account compatibility of the IL-24 protein described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

(A) IL-20 Antagonists

In certain embodiments, the additional pharmaceutically active agent to be co-used with the IL-24 protein for treating or delaying the onset of a liver disease is an interleukin 20 (IL-20) antagonist, which may be an antibody that binds to either IL-20 or an IL-20 receptor and blocks the signaling pathway mediated by IL-20.

IL-20 is a pro-inflammatory cytokine that belongs to the IL-10 cytokine family. The IL-20 described herein refers to interleukin-20 and variants thereof that retain at least part of the activity of IL-20. As used herein, IL-20 includes all mammalian species of native sequence IL-20, including human, canine, feline, equine, or bovine. In one example, the IL-20 is a human IL-20 (GenBank accession no. NP_061194.2).

IL-20 activates the IL-20 signaling pathway via binding to IL-20 receptor, which is a dimeric complex contains subunits IL-20R1 and IL-20R2 (also known as RA and RB). Such an IL-20 receptor is shared by three functionally different cytokines, i.e., IL-19, IL-20, and IL-24, suggesting that this receptor mediates different signaling pathways dependent upon its binding to a specific cytokine. IL-20 is also capable of binding to a dimeric complex containing IL-20R2 and IL-22R1. The IL-20 receptor disclosed herein refers to one or more polypeptides that are capable of binding to and being activated by IL-20. IL-20 receptors disclosed herein include IL-20R1, IL-20R2 and IL-22R1 of any mammalian species, including, but are not limited to, human, canine, feline, equine, primate, or bovine. Examples of human IL-20 receptors include hIL-20R1 (GenBank Accession No. NM_014432.2), hIL-20R2 (GenBank Accession No. NM_144717.2) and hIL-22R1 (NM_181309.1). Sequences of human IL-20 receptors have been described; for example, in U.S. Pat. Nos. 6,610,286; 7,122,632; 7,393,684; and 7,537,761; and U.S. Pat. App. Pub. Nos. 2006/0263850 A1; 2006/0263851 A1; 2008/0247945 A1, and 2009/0074661 A1.

The IL-20 antagonist to be used in the methods described herein is a molecule that blocks, suppresses, or reduces (including significantly) the biological activity of IL-20, including downstream pathways mediated by IL-20 signaling, such as receptor binding and/or elicitation of a cellular response to IL-20. See US2011/0064731, which is incorporated by reference herein in its entirety. The term “antagonist” implies no specific mechanism of biological action whatsoever, and is deemed to expressly include and encompass all possible pharmacological, physiological, and biochemical interactions with IL-20 whether direct or indirect. For purpose of the present disclosure, it will be explicitly understood that the term “antagonist” encompass all the previously identified terms, titles, and functional states and characteristics whereby the IL-20 itself (e.g., human IL-20), an IL-20 biological activity (including but not limited to its ability to mediate any aspect of the target liver disease described herein), or the consequences of the biological activity, are substantially nullified, decreased, or neutralized in any meaningful degree, e.g., by at least 20%, 50%, 70%, 85%, 90%, 100%, 150%, 200%, 300%, or 500%, or by 10-fold, 20-fold, 50-fold, 100-fold, 1000-fold, or 10⁴-fold.

Exemplary IL-20 antagonists include, but are not limited to, an anti-IL-20 antibody, an anti-sense nucleic acid molecule directed to an IL-20 (including an anti-sense nucleic acid directed to a nucleic acid encoding IL-20), a small interfering RNA (siRNA) directed toward an IL-20 nucleic acid, a microRNA directed toward an IL-20 nucleic acid, an IL-20 inhibitory compound, an anti-IL-20R antibody (e.g., an antibody specifically binds IL-20R1, IL-20R2, or the dimeric complex formed thereby), an antisense nucleic acid molecule directed to a subunit of an IL-20 receptor, an siRNA or a microRNA directed to a nucleic acid encoding a subunit of an IL-20 receptor, or an IL-20R inhibitory compound. In some embodiments, an IL-20 antagonist binds IL-20 or IL-20 receptor and prevents the formation of IL-20-IL-20R complex, thereby inhibiting the IL-20 signaling pathway. In other embodiments, an IL-20 antagonist inhibits or reduces IL-20 synthesis and/or production (release). Such antagonists include antisense molecules, siRNAs and microRNAs.

An antibody (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody” encompasses not only intact (i.e., full-length) polyclonal or monoclonal antibodies, but also antigen-binding fragments thereof (such as Fab, Fab′, F(ab′)₂, Fv), single chain (scFv), mutants thereof, fusion proteins comprising an antibody portion, humanized antibodies, chimeric antibodies, diabodies, linear antibodies, single chain antibodies, multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

The antibodies to be used in the methods described herein can be murine, rat, human, or any other origin (including chimeric or humanized antibodies). In some examples, the antibody comprises a modified constant region, such as a constant region that is immunologically inert, e.g., does not trigger complement mediated lysis, or does not stimulate antibody-dependent cell mediated cytotoxicity (ADCC). ADCC activity can be assessed using methods disclosed in U.S. Pat. No. 5,500,362. In other embodiments, the constant region is modified as described in Eur. J. Immunol. (1999) 29:2613-2624; PCT Application No. PCT/GB99/01441; and/or UK Patent Application No. 9809951.8.

Any of the antibodies described herein can be either monoclonal or polyclonal. A “monoclonal antibody” refers to a homogenous antibody population and a “polyclonal antibody” refers to a heterogeneous antibody population. These two terms do not limit the source of an antibody or the manner in which it is made.

In one example, the antibody used in the methods described herein is a humanized antibody. Humanized antibodies refer to forms of non-human (e.g. murine) antibodies that are specific chimeric immunoglobulins, immunoglobulin chains, or antigen-binding fragments thereof that contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, six) which are altered with respect to the original antibody, which are also termed one or more CDRs “derived from” one or more CDRs from the original antibody. Humanized antibodies may also involve affinity maturation.

In another example, the antibody described herein is a chimeric antibody, which can include a heavy constant region and a light constant region from a human antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or the constant region.

In some examples, the antibody disclosed herein specifically binds a target antigen, such as human IL-20 or one of the two subunits of a human IL-20 receptor (e.g., IL-20R1). An antibody that “specifically binds” (used interchangeably herein) to a target or an epitope is a term well understood in the art, and methods to determine such specific binding are also well known in the art. A molecule is said to exhibit “specific binding” if it reacts or associates more frequently, more rapidly, with greater duration and/or with greater affinity with a particular target antigen than it does with alternative targets. An antibody “specifically binds” to a target antigen if it binds with greater affinity, avidity, more readily, and/or with greater duration than it binds to other substances. For example, an antibody that specifically (or preferentially) binds to an IL-20 epitope is an antibody that binds this IL-20 epitope with greater affinity, avidity, more readily, and/or with greater duration than it binds to other IL-20 epitopes or non-IL-20 epitopes. It is also understood by reading this definition that, for example, an antibody that specifically binds to a first target antigen may or may not specifically or preferentially bind to a second target antigen. As such, “specific binding” or “preferential binding” does not necessarily require (although it can include) exclusive binding. Generally, but not necessarily, reference to binding means preferential binding.

(i) Anti-IL-20 Antibodies

Antibodies capable of interfering with the IL-20 signaling pathway can be an antibody that binds an IL-20 (e.g., a human IL-20) and inhibits IL-20 biological activity and/or downstream pathways mediated by IL-20. Alternatively, such antibodies can be antibodies that bind an IL-20 receptor (IL-20R), e.g., bind to one or both of the subunits of the IL-20 receptor, and suppress the downstream signaling pathways mediated by the receptor triggered by IL-20.

An anti-IL-20 antibody is an antibody capable of binding to IL-20 and inhibits IL-20 biological activity and/or downstream pathway(s) mediated by IL-20 signaling. In some examples, an anti-IL-20 antibody used in the methods described herein suppresses the IL-20 signaling pathway by at least 20%, at least 40%, at least 50%, at least 75%, at least 90%, at least 100%, or by at least 2-fold, at least 5-fold, at least 10-fold, at least 20-fold, at least 50-fold, at least 100-fold, or at least 1000-fold. Examples of anti-IL-20 antibodies include, but are not limited to, those disclosed in U.S. Pat. Nos. 7,435,800; 7,115,714; 7,119,175; 7,151,166; and 7,393,684; and PCT publications WO 2007/081465; WO 99/27103; WO 2004/085475; and WO 2005052000.

The binding affinity of an anti-IL-20 antibody to IL-20 (such as human IL-20) can be less than any of about 100 nM, about 50 nM, about 10 nM, about 1 nM, about 500 pM, about 100 pM, or about 50 pM to any of about 2 pM. Binding affinity can be expressed K_{D} or dissociation constant, and an increased binding affinity corresponds to a decreased K_{D}. One way of determining binding affinity of antibodies to IL-20 is by measuring binding affinity of monofunctional Fab fragments of the antibody. To obtain monofunctional Fab fragments, an antibody (for example, IgG) can be cleaved with papain or expressed recombinantly. The affinity of an anti-IL-20 Fab fragment of an antibody can be determined by surface plasmon resonance (BIAcore3000™ surface plasmon resonance (SPR) system, BIAcore, INC, Piscaway N.J.). Kinetic association rates (kₒₙ) and dissociation rates (k_{off}) (generally measured at 25° C.) are obtained; and equilibrium dissociation constant (K_{D}) values are calculated as k_{off}/kₒₙ.

In some embodiments, the antibody binds human IL-20, and does not significantly bind an IL-20 from another mammalian species. In some embodiments, the antibody binds human IL-20 as well as one or more IL-20 from another mammalian species. In still other embodiments, the antibody binds IL-20 and does not significantly cross-react with other cytokines (such as the related cytokines IL-10, IL-17A, IL-19, IL-22, IL-24 and IL-26). The epitope(s) bound by the antibody can be continuous or discontinuous.

In some embodiments, the anti-IL-20 antibody described herein is anti-IL-20 antibody 7E, which refers to monoclonal antibody mAb 7E and its functional variants. MAb 7E is produced by the hybridoma cell line deposited at the American Type Culture Collection, 10801 University Boulevard, Manassas, Va. 20110-2209, U.S.A. and assigned a deposit number PTA-8687. This hybridoma cell line will be released to the public irrevocably and without restriction/condition upon granting a US patent on this application, and will be maintained in the ATCC for a period of at least 30 years from the date of the deposit for the enforceable life of the patent or for a period of 5 years after the date of the most recent. See also U.S. Pat. Nos. 8,206,712 and 7,611,705, the relevant disclosures of each of which are incorporated by reference herein.

The amino acid sequences and encoding nucleotide sequences of the heavy chain variable region (V_{H}) and light chain variable region (V_{L}) of mAb7E are produced below:

### Nucleotide Sequence (SEQ ID NO:1) and Amino Acid Sequence (SEQ ID NO:2) of mAb 7E Heavy Chain Variable Region

Nucleotide sequence (SEQ ID NO: 1) and amino acid sequence
(SEQ ID NO: 2) of mAb 7E heavy chain variable region

| | |
|---|---|
| gaa ttg aag ctt gag gag tct gga gga ggc ttg gtg cag cct gga | 45 |
| E L K L E E S G G G L V Q P G | 15 |
| gga tcc atg aaa ctc tct tgt gct gcc tct gga ttc act ttt agt | 90 |
| G S M K L S C A A S G F T F S | 30 |
| gac gcc tgg atg gac tgg gtc cgc cag tct cca gag aag ggg ctt | 135 |
| D A W M D W V R Q S P E K G L | 45 |
| gag tgg att gct gaa att aga agc aaa gct aat aat tat gca aca | 180 |
| E W I A E I R S K A N N Y A T | 60 |
| tac ttt gct gag tct gtg aaa ggg agg ttc acc atc tca aga gat | 215 |
| Y F A E S V K G R F T I S R D | 75 |
| gat tcc aaa agt ggt gtc tac ctg caa atg aac aac tta aga gct | 270 |
| D S K S G V Y L Q M N N L R A | 90 |
| gag gac act ggc att tat ttc tgt acc aag tta tca cta cgt tac | 315 |
| E D T G I Y F C T K L S L R Y | 105 |
| tgg ttc ttc gat gtc tgg ggc gca ggg acc acg gtc acc gtc tcc | 360 |
| W F F D V W G A G T T V T V S | 120 |
| tca | 363 |
| S | 121 |

Nucleotide sequence (SEQ ID NO: 3) and amino acid sequence
(SEQ ID NO: 4) of mAb 7E light chain variable region

| | |
|---|---|
| gat ttt gtg atg acc cag act cca ctc act ttg tcg gtt acc att | 45 |
| D F V M T Q T P L T L S V T I | 15 |
| gga caa cca gcc tcc atc tct tgc aag tca agt cag agc ctc ttg | 90 |
| G Q P A S I S C K S S Q S L L | 30 |
| gat agt gat gga aag aca tat ttg aat tgg ttg tta cag agg cca | 135 |
| D S D G K T Y L N W L L Q R P | 45 |
| ggc cag tct cca aag cac ctc atc tat ctg gtg tct aaa ctg gac | 180 |
| G Q S P K H L I Y L V S K L D | 60 |
| tct gga gtc cct gac agg ttc act ggc agt gga tca ggg acc gat | 215 |
| S G V P D R F T G S G S G T D | 75 |
| ttc aca ctg aga atc agc aga gtg gag gct gag gat ttg gga gtt | 270 |
| F T L R I S R V E A E D L G V | 90 |
| tat tat tgc tgg caa agt aca cat ttt ccg tgg acg ttc ggt gga | 315 |
| Y Y C W Q S T H F P W T F G G | 105 |
| ggc acc aag ctg gaa atc aaa cgg | 339 |
| G T K L E I K R | 113 |

A functional variant (equivalent) of mAb7E has essentially the same epitope-binding specificity as mAb7E and exhibits at least 20% (e.g., 30%, 40%, 50%, 60%, 70%, 80%, 90%, or greater) of the activity of neutralizing a signaling pathway mediated by IL-20 as relative to mAb7E. In some embodiments, a functional variant of mAb7E contains the same regions/residues responsible for antigen-binding as mAb7E, such as the same specificity-determining residues in the CDRs or the whole CDRs. The regions/residues that are responsible for antigen-binding can be identified from amino acid sequences of the heavy chain/light chain sequences of mAb7GW or mAb51D (shown above) by methods known in the art. See, e.g., www.bioinf.org.uk/abs; Almagro, J. Mol. Recognit. 17:132-143 (2004); and Chothia et al., J. Mol. Biol. 227:799-817 (1987).

In addition, determination of CDR regions in an antibody is well within the skill of the art. There are at least two techniques for determining CDRs: (1) an approach based on cross-species sequence variability (i.e., Kabat et al. Sequences of Proteins of Immunological Interest, (5th ed., 1991, National Institutes of Health, Bethesda Md.)); and (2) an approach based on crystallographic studies of antigen-antibody complexes (Chothia et al. (1989) Nature 342:877; Al-lazikani et al (1997) J. Molec. Biol. 273:927-948)). As used herein, a CDR may refer to CDRs defined by either approach or by a combination of both approaches.

In some examples, a functional variant of mAb7E comprises a V_{H} chain that includes a V_{H} CDR1, V_{H} CDR2, and V_{H} CDR3 at least 75% (e.g., 80%, 85%, 90%, 95%, or 98%) identical to the corresponding V_{H} CDRs of mAb7E, and a V_{L} chain that includes a V_{L} CDR1, V_{L} CDR2, and V_{L} CDR3 at least 75% (e.g., 80%, 85%, 90%, 95%, or 98%) identical to the corresponding V_{H} CDRs of mAb7E.

Alternatively, the functional variant of mAb7E comprises a V_{H} chain at least 75% (e.g., 80%, 85%, 90%, 95%, or 98%) identical to the V_{H} chain (mature or precursor) of mAb7E and a V_{L} chain at least 75% (e.g., 80%, 85%, 90%, 95%, or 98%) identical to the V_{L} chain (mature of precursor) of mAb7E.

The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

In other examples, a functional variant of mAb7E comprises a V_{H} chain that includes up to 5 (e.g., 1, 2, 3, 4, or 5) amino acid residue variations in the V_{H} CDR regions (V_{H} CDR1, CDR2, and/or CDR3) as compared to the V_{H} CDRs of mAb7E, and/or a V_{L} chain that includes up to 5 (e.g., 1, 2, 3, 4, or 5) amino acid residue variations in the V_{L} CDR regions (V_{L} CDR1, CDR2, and/or CDR3) as compared to the V_{H} CDRs of mAb7E.

Functional variants of mAb7E are also disclosed in U.S. Pat. No. 7,611,705 and US2011/0064731, both of which are incorporated by reference herein.

In one example, a functional variant of mAb7E is a humanized antibody derived from mAb7E. Provided below are exemplary humanized mAb7E antibodies HL1 and HL2; see also U.S. Pat. No. 8,597,647, the relevant disclosures therein are incorporated by reference.

### Amino Acid Sequence and Encoding Nucleotide Sequence of the V_{H} Chain of Humanized Anti-IL-20 Antibodies HL1 and HL2:

```
(SEQ ID NO: 5)
                                ATG TAC TTG GGA CTG AAC TAT GTT
(SEQ ID NO: 6)
                                  M Y L G L N Y V
TTC ATC GTT TTT CTC CTG AAT GGT GTC CAG AGT GAA
  F I V F L L N G V Q S E
GTG CAG CTT GTG GAG TCT GGA GGA GGC TTG GTG CAG
  V Q L V E S G G G L V Q
CCT GGA GGA TCC CTG AAA CTC TCT TGT GCT GCC TCT
  P G G S L K L S C A A S
GGA TTC ACT TTT AGT GAC GCC TGG ATG GAC TGG GTC
  G F T F S [symbol] [symbol] [symbol] [symbol] [symbol]           W V
CGC CAG GCT TCC GGG AAG GGG CTT GAG TGG ATT GCT
  R Q A S G K G L E W I A
GAA ATT AGA AGC AAA GCT AAT AAT TAT GCA ACA TAC

[symbol]

[symbol]

[symbol]

[symbol]

[symbol]

[symbol]

[symbol]

[symbol]

[symbol]

[symbol]

[symbol]

[symbol]

TTT GCT GAG TCT GTG AAA GGG AGG TTC ACC ATC TCA [symbol] [symbol] [symbol] [symbol] [symbol] [symbol] [symbol]
                              R F T I S
AGA GAT GAT TCC AAA AAC ACC GCC TAC CTG CAA ATG
  R D D S K N T A Y L Q M
AAC AGC TTA AAA ACC GAG GAC ACT GCC GTT TAT TAC
  N S L K T E D T A V Y Y
TGT ACC AAG TTA TCA CTG CGT TAC TGG TTC TTC GAT
  C T K [symbol] [symbol] [symbol] [symbol] [symbol] [symbol] [symbol] [symbol] [symbol]
GTC TGG GGC CAG GGG ACC CTG GTC ACC GTC TCC TCA [symbol]
    W G Q G T L V T V S S
```

The underlined region refers to the signal peptide and the boldfaced/italic regions are the CDRs. SEQ ID NOs: 8 and 7 represent the mature V_{H} amino acid sequence (lacking the signal peptide) and its encoding nucleotide sequence, respectively.

### Amino Acid Sequence and Encoding Nucleotide Sequence of the V_{L} Chain (VL2) of a Humanized Anti-IL-20 Antibody HL2:

```
(SEQ ID NO: 9)
                        ATG ATG AGT CCT GCC CAG TTC CTG TTT
(SEQ ID NO: 10)
                          M M S P A Q F L F
CTG TTG GTG CTC TGG ATT CGG GAA ACC AAC GGT GAT
  L L V L W I R E T N G D
ATC GTG ATG ACC CAG ACT CCA CTC TCT TTG TCC GTT [symbol]
      V M T Q T P L S L S V
ACC CCT GGA CAA CCA GCC TCC ATC TCT TGC AAG TCA
  T P G Q P A S I S C [symbol] [symbol]
AGT CAG AGC CTC TTG GAT AGT GAT GGA AAG ACA TAT

[symbol]

[symbol]

[symbol]

[symbol]

[symbol]

[symbol]

[symbol]

[symbol]

[symbol]

[symbol]

[symbol]

[symbol]

TTG AAT TGG TTG TTA CAG AAG CCA GGC CAG TCT CCA [symbol] [symbol]
      W L L Q K P G Q S P
CAG CAC CTC ATC TAT CTG GTG TCT AAA CTG GAC TCT
  Q H L I Y [symbol] [symbol] [symbol] [symbol] [symbol] [symbol] [symbol]
GGA GTC CCT GAC AGG TTC AGT GGC AGT GGA TCA GGG
  G V P D R F S G S G S G
ACC GAT TTC ACA CTG AAA ATC AGC AGA GTG GAG GCT
  T D F T L K I S R V E A
GAG GAT GTT GGA GTT TAT TAT TGC TGG CAA AGT ACA
  E D V G V Y Y C [symbol] [symbol] [symbol] [symbol]
CAT TTT CCC TGG ACC TTC GGT GGA GGC ACC AAG GTG [symbol] [symbol] [symbol] [symbol] [symbol]
                      F G G G T K V
GAA ATC AAA
  E I K
```

The underlined region refers to the signal peptide and the boldfaced/italic regions are the CDRs. SEQ ID NOs: 12 and 11 represent the mature V_{L} amino acid sequence (lacking the signal peptide) and its encoding nucleotide sequence, respectively.

Humanized antibody HL1 comprises the same V_{H} chain as HL2 and a V_{L} chain (SEQ ID NO:13; mature form) that is otherwise identical to the V_{L} of HL2 except that the I residue at position 2 of mature V_{L} of HL2 is replaced with F.

Also disclosed herein are functional variants of the above-noted humanized antibodies HL1 and HL2. Such functional variants can comprise a V_{H} chain that comprises an amino acid sequence at least 85% (e.g., 90%, 92%, 94%, 95%, 96%, 97%, 98%, or 99%) identical to that of the V_{H} of HL1 and HL2 (precursor or mature form; SEQ ID NO:6 and SEQ ID NO:8, respectively) and a V_{L} chain that has an amino acid sequence at least 85% (e.g., 90%, 92%, 94%, 95%, 96%, 97%, 98%, or 99%) identical to that of the V_{L} of HL2 (precursor or mature form; SEQ ID NO:10 and SEQ ID NO:12, respectively). These variants are capable of binding to an IL-20 molecule, particularly a human IL-20 molecule. In some examples, the variants possess similar antigen-binding affinity relative to the exemplary humanized antibody described above (e.g., having a K_{d}<4×10).

(ii) Anti-IL-20R Antibodies

An anti-IL-20R antibody is an antibody capable of binding to an IL-20R (e.g., binding to either one of its two subunits or binding to the dimeric complex) and inhibits the biological activity of the IL-20R and/or its downstream pathway(s) mediated by IL-20. In some examples, an anti-IL-20 antibody used in the methods described herein suppresses the IL-20 signaling pathway by at least 20%, at least 40%, at least 50%, at least 75%, at least 90%, at least 100%, or by at least 2-fold, at least 5-fold, at least 10-fold, at least 20-fold, at least 50-fold, at least 100-fold, or at least 1000-fold. In some examples, the anti-IL-20R antibody specifically binds IL-20R1, such as human IL-20R1. Such an antibody may have low affinity to IL-20R2 or the IL-20R1/IL-20R2 complex or does not bind IL-20R2 or the IL-20R1/IL-20R2 complex. In other examples, the anti-IL-20R antibody specifically binds IL-20R2, such as human IL-20R2. Such an antibody may have low affinity to IL-20R1 or the IL-20R1/IL-20R2 complex or does not bind IL-20R1 or the IL-20R1/IL-20R2 complex. In yet other examples, the anti-IL-20R antibody described herein specifically binds the IL-20R1/IL-20R2 complex.

The binding affinity of an anti-IL-20R antibody to IL-20R or a subunit thereof (such as human IL-20R or human IL-20R1) can be less than any of about 100 nM, about 50 nM, about 10 nM, about 1 nM, about 500 pM, about 100 pM, or about 50 pM to any of about 2 pM. Binding affinity can be expressed K_{D} or dissociation constant, and an increased binding affinity corresponds to a decreased K_{D}. One way of determining binding affinity of antibodies to IL-20R is by measuring binding affinity of monofunctional Fab fragments of the antibody. To obtain monofunctional Fab fragments, an antibody (for example, IgG) can be cleaved with papain or expressed recombinantly. The affinity of an anti-IL-20R Fab fragment of an antibody can be determined by surface plasmon resonance (BIAcore3000™ surface plasmon resonance (SPR) system, BIAcore, INC, Piscaway N.J.). Kinetic association rates (kₒₙ) and dissociation rates (k_{off}) (generally measured at 25° C.) are obtained; and equilibrium dissociation constant (K_{D}) values are calculated as k_{off}/kₒₙ.

In some embodiments, the antibody binds human IL-20R or a subunit thereof (e.g., human IL-20R1), and does not significantly bind an IL-20R from another mammalian species. In some embodiments, the antibody binds human IL-20R as well as one or more IL-20R from another mammalian species. In still other embodiments, the antibody binds IL-20R and does not significantly cross-react with other cytokine receptors. The epitope(s) bound by the antibody can be continuous or discontinuous.

In some embodiments, the antibody used in the methods described herein is an antibody having the same heavy chain and light chain variable regions (V_{H} and V_{L}) as those of monoclonal antibody mAb7GW or mAb51D, the monoclonal antibodies, an antigen-binding fragment thereof, or a functional equivalent of either mAb7GW or mAb51D. US2011/0256093, which is herein incorporated by reference in its entirety. Shown below are the amino acid sequences of the heavy chains and light chains of mAb7GW and mAb51D, as well as their encoding nucleotide sequences.

```
Heavy Chain of mAb7GW:
Amino Acid Sequence
(SEQ ID NO: 14)
M R V L I L L W L F T A F P G I L S V V Q L Q E S G P G L V K P S Q S L S L T C T V T G Y S I
            Signal peptide
T S D Y A W N W I R Q F P G N R L E W M G Y I D Y S G S T K Y N P S L K S R I S V T R D
    CDR1 CDR2
T S K N Q F F L Q L N S V T T E D T A T Y Y C A R D F G D A Y W G Q G T L V T V S A A K
                                                    CDR3
T T P P S V Y P L A P G S A A Q T N S M V T L G C L V K G Y F P E P V T V T W N S G S L S S G V H
T F P A V L Q S D L Y T L S S S V T V P S S T W P S E T V T C N V A H P A S S T K V D K K I V P R D
C G C K P C I C T V P E V S S V F I F P P K P K D V L T I T L T P K V T C V V V D I S K D D P E V Q
F S W F V D D V E V H T A Q T Q P R E E Q F N S T F R S V S E L P I M H Q D W L N G K E F K C R
V N S A A F P A P I E K T I S K T K G R P K A P Q V Y T I P P P K E Q M A K D K V S L T C M I T D F
F P E D I T V E W Q W N G Q P A E N Y K N T Q P I M D T D G S Y F V Y S K L N V Q K S N W E A G
N T F T C S V L H E G L H N H H T E K S L S H S P G K
(The italic region refers to the heavy chain constant region.)
Nucleotide Sequence
(SEQ ID NO: 15)
ATGAGAGTGCTGATTCTTTTGTGGCTGTTCACAGCCTTTCCTGGTATCCTGTCTGTTGTGCAGC
          Signal peptide
TTCAGGAGTCGGGACCTGGCCTGGTGAAACCTTCTCAGTCTCTGTCCCTCACCTGCACTGTCA
CTGGCTACTCAATCACCAGTGATTATGCCTGGAACTGGATCCGGCAGTTTCCAGGA
                                                CDR1
AACAGACTGGAGTGGATGGGCTACATAGACTACAGTGGTAGCACTAAATACAACCCC
                                                                          CDR2
TCTCTCAAAAGTCGAATCTCTGTCACTCGAGACACATCCAAGAACCAGTTCTTCCTGCAGTT
GAATTCTGTGACTACTGAGGACACAGCCACATATTACTGTGCAAGAGACTTTGGTG
                                                                                                        CDR3
ATGCTTACTGGGGCCAGGGGACTCTGGTCACTGTCTCTGCAGCCAAAACGACACCCCCATCTG
TCTATCCACTGGCCCCTGGATCTGCTGCCCAAACTAACTCCATGGTGACCCTGGGATGCCTGGTCA
AGGGCTATTTCCCTGAGCCAGTGACAGTGACCTGGAACTCTGGATCCCTGTCCAGCGGTGTGCAC
ACCTTCCCAGCTGTCCTGCAGTCTGACCTCTACACTCTGAGCAGCTCAGTGACTGTCCCCTCCAGC
ACCTGGCCCAGCGAGACCGTCACCTGCAACGTTGCCCACCCGGCCAGCAGCACCAAGGTGGACA
AGAAAATTGTGCCCAGGGATTGTGGTTGTAAGCCTTGCATATGTACAGTCCCAGAAGTATCATCTGT
CTTCATCTTCCCCCCAAAGCCCAAGGATGTGCTCACCATTACTCTGACTCCTAAGGTCACGTGTGTT
GTGGTAGACATCAGCAAGGATGATCCCGAGGTCCAGTTCAGCTGGTTTGTAGATGATGTGGAGGT
GCACACAGCTCAAACGCAACCCCGGGAGGAGCAGTTCAACAGCACTTTCCGCTCAGTCAGTGAAC
TTCCCATCATGCACCAGGACTGGCTCAATGGCAAGGAGTTCAAATGCAGGGTCAACAGTGCAGCTT
TCCCTGCCCCCATCGAGAAAACCATCTCCAAAACCAAAGGCAGACCGAAGGCTCCACAGGTGTAC
ACCATTCCACCTCCCAAGGAGCAAATGGCCAAGGATAAAGTCAGTCTGACCTGCATGATAACAGAC
TTCTTCCCTGAAGACATTACTGTGGAGTGGCAGTGGAATGGGCAGCCAGCGGAGAACTACAAGAA
CACTCAGCCCATCATGGACACAGATGGCTCTTACTTCGTCTACAGCAAGCTCAATGTGCAGAAGAG
CAACTGGGAGGCAGGAAATACTTTCACCTGCTCTGTGTTACATGAGGGCCTGCACAACCACCATAC
TGAGAAGAGCCTCTCCCACTCTCCTGGTAAATGA
(The italic region encodes the heavy chain constant region.)
Light Chain of mAb7GW:
Amino Acid Sequence
(SEQ ID NO: 16)
M D S Q A Q V L M L L L L W V S G S C G D I V M S Q S P S S L A V S V G E K V T M S C K S S
            Signal peptide
Q S L L Y S R N Q K N Y L A W Y Q L K P G Q S P K L L I Y W A S T R E S G V P D R F T G
              CDR1 CDR2
S G S G T D F T L T I S S V K A E D L A V Y Y C Q Q Y Y S Y P L T F G A G T K L E L K R A
                                                                                                      CDR3
D A A P T V S I F P P S S E Q L T S G G A S V V C F L N N F Y P K D I N V K W K I D G S E R Q N G
V L N S W T D Q D S K D S T Y S M S S T L T L T K D E Y E R H N S Y T C E A T H K T S T S P I V K S
F N R N E C
(The italic region refers to the light chain constant region.)
Nucleotide Sequence
(SEQ ID NO: 17)
ATGGATTCACAGGCCCAGGTTCTTATGTTACTGCTGCTATGGGTATCTGGTTCCTGTGGGGACA
                Signal peptide
TTGTGATGTCACAGTCTCCATCCTCCCTAGCTGTGTCAGTTGGAGAGAAGGTTACTATGAGCT
GCAAGTCCAGTCAGAGCCTTTTATATAGTAGGAATCAAAAGAACTACTTGGCCT
                                            CDR1
GGTACCAGCTGAAGCCAGGGCAGTCTCCTAAACTGCTGATTTACTGGGCATCCACTAGG
                                                                                                CDR2
GAATCTGGGGTCCCTGATCGCTTCACAGGCAGTGGATCTGGGACAGATTTCACTCTCACCAT
CAGCAGTGTGAAGGCTGAAGACCTGGCAGTTTATTACTGTCAGCAATATTATAGCTA
                                                                                              CDR3
TCCGCTCACGTTCGGTGCTGGGACCAAGCTGGAGCTGAAACGGGCTGATGCTGCACCAACTG
TATCCATCTTCCCACCATCCAGTGAGCAGTTAACATCTGGAGGTGCCTCAGTCGTGTGCTTCTTGAA
CAACTTCTACCCCAAAGACATCAATGTCAAGTGGAAGATTGATGGCAGTGAACGACAAAATGGCGT
CCTGAACAGTTGGACTGATCAGGACAGCAAAGACAGCACCTACAGCATGAGCAGCACCCTCACGT
TGACCAAGGACGAGTATGAACGACATAACAGCTATACCTGTGAGGCCACTCACAAGACATCAACTT
CACCCATTGTCAAGAGCTTCAACAGGAATGAGTGTTAG
(The italic region encodes the light chain constant region.)
Heavy Chain of mAb51D:
Amino Acid Sequence
(SEQ ID NO: 18)
MNFGLSLIFLALILKGVQCEVQLVEAGGDLVKPGGSLKLSCAASGFSLSNYGMSWVRQTPDK
          Signal peptide CDR1
RLEWVASISSGGRFTSYPDSVRGRFTISRDNAKNTLYLQMSGLKSEDTAMYYCARHDGNG
                    CDR2 CDR3
GDYWGQGTSVTVSSAKTTPPSVYPLAPGSAAQTNSMVTLGCLVKGYFPEPVTVTWNSGSLSSGVHTF
PAVLQSDLYTLSSSVTVPSSTWPSETVTCNVAHPASSTKVDKKIVPRDCGCKPCICTVPEVSSVFIFPPKP
KDVLTITLTPKVTCVVVDISKDDPEVQFSWFVDDVEVHTAQTQPREEQFNSTFRSVSELPIMHQDWLN
GKEFKCRVNSAAFPAPIEKTISKTKGRPKAPQVYTIPPPKEQMAKDKVSLTCMITDFFPEDITVEWQWN
GQPAENYKNTQPIMDTDGSYFVYSKLNVQKSNWEAGNTFTCSVLHEGLHNHHTEKSLSHSPGK
(The italic region refers to the heavy chain constant region.)
Nucleotide Sequence
(SEQ ID NO: 19)
ATGAACTTCGGGCTCAGCCTGATTTTCCTTGCCCTCATTTTAAAAGGTGTCCAGTGTGAGGTGC
                    Signal peptide
AGCTGGTGGAGGCTGGGGGAGACTTAGTGAAGCCTGGAGGGTCCCTGAAACTCTCCTGTGC
GGCCTCTGGATTCAGTTTGAGTAACTATGGCATGTCCTGGGTTCGCCAGACTCCAGA
                                                          CDR1
CAAGAGGCTGGAGTGGGTCGCAAGCATTAGTAGTGGTGGTCGTTTCACCTCCTATCC
                                                                                    CDR2
AGACAGTGTGAGGGGGCGATTCACCATCTCCAGAGACAATGCCAAGAACACCCTGTACCT
GCAAATGAGCGGTCTGAAGTCTGAGGACACAGCCATGTATTACTGTGCAAGACACGACGGC
AACGGTGGGGACTACTGGGGTCAAGGAACCTCAGTCACCGTCTCCTCAGCCAAA
        CDR3
ACGACACCCCCATCTGTCTATCCACTGGCCCCTGGATCTGCTGCCCAAACTAACTCCATGGTGACC
CTGGGATGCCTGGTCAAGGGCTATTTCCCTGAGCCAGTGACAGTGACCTGGAACTCTGGATCCCT
GTCCAGCGGTGTGCACACCTTCCCAGCTGTCCTGCAGTCTGACCTCTACACTCTGAGCAGCTCAGT
GACTGTCCCCTCCAGCACCTGGCCCAGCGAGACCGTCACCTGCAACGTTGCCCACCCGGCCAGC
AGCACCAAGGTGGACAAGAAAATTGTGCCCAGGGATTGTGGTTGTAAGCCTTGCATATGTACAGTC
CCAGAAGTATCATCTGTCTTCATCTTCCCCCCAAAGCCCAAGGATGTGCTCACCATTACTCTGACTC
CTAAGGTCACGTGTGTTGTGGTAGACATCAGCAAGGATGATCCCGAGGTCCAGTTCAGCTGGTTTG
TAGATGATGTGGAGGTGCACACAGCTCAGACGCAACCCCGGGAGGAGCAGTTCAACAGCACTTTC
CGCTCAGTCAGTGAACTTCCCATCATGCACCAGGACTGGCTCAATGGCAAGGAGTTCAAATGCAGG
GTCAACAGTGCAGCTTTCCCTGCCCCCATCGAGAAAACCATCTCCAAAACCAAAGGCAGACCGAAG
GCTCCACAGGTGTACACCATTCCACCTCCCAAGGAGCAGATGGCCAAGGATAAAGTCAGTCTGAC
CTGCATGATAACAGACTTCTTCCCTGAAGACATTACTGTGGAGTGGCAGTGGAATGGGCAGCCAGC
GGAGAACTACAAGAACACTCAGCCCATCATGGACACAGATGGCTCTTACTTCGTCTACAGCAAGCT
CAATGTGCAGAAGAGCAACTGGGAGGCAGGAAATACTTTCACCTGCTCTGTGTTACATGAGGGCCT
GCACAACCACCATACTGAGAAGAGCCTCTCCCACTCTCCTGGTAAATGA
(The italic region encodes the heavy chain constant region.)
```

Light Chain of mAb51D:

```
Amino Acid Sequence
(SEQ ID NO: 20)
MDFQVQIFSFLLISASVIMSRGQIVLSQFPAILSASPGEKVTMTCRARSS
          Signal peptide CDR1
VSFMHWYQQKPGS
SPKPWIYATSNLASGVPPRFSGSGSGTSYSLTISRVEAEDAATYYCQQWS
              CDR2 CDR3
SNPYTFGGGTKLE
IKRADAAPTVSIFPPSSEQLTSGGASVVCFLNNFYPKDINVKWKIDGSER
QNGVLNSWTDQDSKDSTYSMSSTLTLTKDEYERHNSYTCEATHKTSTSPI
VKSFNRNEC
(The italic region refers to the light chain
constant region)
Nucleotide Sequence
(SEQ ID NO: 21)
ATGGATTTTCAAGTGCAGATTTTCAGCTTCCTGCTAATCAGTGCTTCAGT
                                    Signal peptide
CATAATGTCCA
GAGGACAAATTGTTCTCTCCCAGTTTCCAGCAATCCTGTCTGCATCTCCA
GGGGAGAAGGTCACAATGACTTGCAGGGCCAGGTCAAGTGTAAGTTTCAT
                                                                      CDR1
GCACTGGTACCAGCAGAA
GCCAGGATCCTCCCCCAAACCCTGGATTTATGCCACATCCAACCTGGCTT
                                                                            CDR2
CTGGAGTCC
CTCCTCGCTTCAGTGGCAGTGGGTCTGGGACCTCTTACTCTCTCACAATC
AGCAGAGTGGAGGCTGAAGATGCTGCCACTTATTACTGCCAGCAGTGGAG
TAGTAACCCATACACGTTC
CDR3
GGAGGGGGGACTAAGCTGGAAATAAAACGGGCTGATGCTGCACCAACTGT
ATCCATCTTCCCACCATCCAGTGAGCAGTTAACATCTGGAGGTGCCTCAG
TCGTGTGCTTCTTGAACAACTTCTACCCCAAAGACATCAATGTCAAGTGG
AAGATTGATGGCAGTGAACGACAAAATGGCGTCCTGAACAGTTGGACTGA
TCAGGACAGCAAAGACAGCACCTACAGCATGAGCAGCACCCTCACGTTGA
CCAAGGACGAGTATGAACGACATAACAGCTATACCTGTGAGGCCACTCAC
AAGACATCAACTTCACCCATTGTCAAGAGCTTCAACAGGAATGAGTGTTA
G
(The italic region encodes the light chain
constant region.)
```

A functional equivalent of mAb7GW or mAb51D has the same epitope-binding specificity as mAb7GW or mAb51D and exhibits at least 20% (e.g., 30%, 40%, 50%, 60%, 70%, 80%, 90%, or greater) of the activity of neutralizing a signaling pathway mediated by IL-20R1 as relative to mAb7GW or mAb51D. In some embodiments, a functional equivalent of mAb7GW or mAb51D contains the same regions/residues responsible for antigen-binding as mAb7GW or mAb51D, such as the same specificity-determining residues in the CDRs or the whole CDRs. The regions/residues that are responsible for antigen-binding can be identified from amino acid sequences of the heavy chain/light chain sequences of mAb7GW or mAb51D (shown above) by methods known in the art. See, e.g., www.bioinf.org.uk/abs; Almagro, J. Mol. Recognit. 17:132-143 (2004); and Chothia et al., J. Mol. Biol. 227:799-817 (1987).

In some examples, a functional equivalent (variant) of mAb7GW or mAb51D comprises a V_{H} chain that includes a V_{H} CDR1, V_{H} CDR2, and V_{H} CDR3 at least 75% (e.g., 80%, 85%, 90%, 95%, or 98%) identical to the corresponding V_{H} CDRs of mAb7GW or mAb51D, and a V_{L} chain that includes a V_{L} CDR1, V_{L} CDR2, and V_{L} CDR3 at least 75% (e.g., 80%, 85%, 90%, 95%, or 98%) identical to the corresponding V_{H} CDRs of mAb7GW or mAb51D.

Alternatively, the functional equivalent of mAb7GW or mAb51D comprises a V_{H} chain at least 75% (e.g., 80%, 85%, 90%, 95%, or 98%) identical to the V_{H} chain (mature or precursor) of mAb7GW or mAb51D and a V_{L} chain at least 75% (e.g., 80%, 85%, 90%, 95%, or 98%) identical to the V_{L} chain (mature of precursor) of mAb7GW or mAb51D.

In other examples, a functional equivalent of mAb7GW or mAb51D comprises a V_{H} chain that includes up to 5 (e.g., 1, 2, 3, 4, or 5) amino acid residue variations in the V_{H} CDR regions (V_{H} CDR1, CDR2, and/or CDR3) as compared to the V_{H} CDRs of mAb7GW or mAb51D, and/or a V_{L} chain that includes up to 5 (e.g., 1, 2, 3, 4, or 5) amino acid residue variations in the V_{L} CDR regions (V_{L} CDR1, CDR2, and/or CDR3) as compared to the V_{H} CDRs of mAb7GW or mAb51D.

Any of the anti-IL-20 or anti-IL-20R antibodies can be prepared by conventional methods (e.g., hybridoma technology or recombinant technology), which are well known in the art. See, for example, Harlow and Lane, (1988) Antibodies: A Laboratory Manual, Cold Spring Harbor Laboratory, New York; Kohler, B. and Milstein, C. (1975) Nature 256:495-497 or as modified by Buck, D. W., et al., In Vitro, 18:377-381 (1982); U.S. Pat. Nos. 5,565,332; 5,580,717; 5,733,743; and 6,265,150; and Winter et al., (1994) Annu. Rev. Immunol. 12:433-455; McCafferty et al., (1990) Nature 348:552-553; Morrison et al. (1984) Proc. Natl. Acad. Sci. USA 81, 6851; Neuberger et al. (1984) Nature 312, 604; and Takeda et al. (1984) Nature 314:452; Queen et al., Proc. Natl. Acad. Sci. USA, 86:10029-10033 (1989); U.S. Pat. Nos. 4,946,778 and 4,704,692; Chapter 11 of Harlow and Lane, Using Antibodies, a Laboratory Manual, Cold Spring Harbor Laboratory Press, Cold Spring Harbor, N.Y., 1999.

(iii) Other IL-20 Antagonists

IL-20 antagonists other than antibodies capable of interfering with the IL-20 signaling pathway as described above can be used in the methods described herein.

In some embodiments of the invention, the IL-20 antagonist comprises at least one antisense nucleic acid molecule capable of blocking or decreasing the expression of a functional IL-20 (e.g., a human IL-20) or a subunit of an IL-20 receptor (e.g., IL-20R1). Nucleotide sequences of the IL-20 and IL-20 receptor subunits are known and are readily available from publicly available databases. See above disclosures. It is routine to prepare antisense oligonucleotide molecules that will specifically bind a target mRNA without cross-reacting with other polynucleotides. Exemplary sites of targeting include, but are not limited to, the initiation codon, the 5′ regulatory regions, the coding sequence and the 3′ untranslated region. In some embodiments, the oligonucleotides are about 10 to 100 nucleotides in length, about 15 to 50 nucleotides in length, about 18 to 25 nucleotides in length, or more. The oligonucleotides can comprise backbone modifications such as, for example, phosphorothioate linkages, and 2′-0 sugar modifications well known in the art.

Alternatively, IL-20/IL-20R expression and/or release can be decreased using gene knockdown, morpholino oligonucleotides, small interfering RNA (siRNA or RNAi), microRNA or ribozymes, methods that are well-known in the art. RNA interference (RNAi) is a process in which a dsRNA directs homologous sequence-specific degradation of messenger RNA. In mammalian cells, RNAi can be triggered by 21-nucleotide duplexes of small interfering RNA (siRNA) without activating the host interferon response. The dsRNA used in the methods disclosed herein can be a siRNA (containing two separate and complementary RNA chains) or a short hairpin RNA (i.e., a RNA chain forming a tight hairpin structure), both of which can be designed based on the sequence of the target gene. Alternatively, it can be a microRNA.

Optionally, a nucleic acid molecule to be used in the method described herein (e.g., an antisense nucleic acid, a small interfering RNA, or a microRNA) as described above contains non-naturally-occurring nucleobases, sugars, or covalent internucleoside linkages (backbones). Such a modified oligonucleotide confers desirable properties such as enhanced cellular uptake, improved affinity to the target nucleic acid, and increased in vivo stability.

In one example, the nucleic acid has a modified backbone, including those that retain a phosphorus atom (see, e.g., U.S. Pat. Nos. 3,687,808; 4,469,863; 5,321,131; 5,399,676; and 5,625,050) and those that do not have a phosphorus atom (see, e.g., U.S. Pat. Nos. 5,034,506; 5,166,315; and 5,792,608). Examples of phosphorus-containing modified backbones include, but are not limited to, phosphorothioates, chiral phosphorothioates, phosphorodithioates, phosphotriesters, aminoalkyl-phosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having 3′-5′ linkages, or 2′-5′ linkages. Such backbones also include those having inverted polarity, i.e., 3′ to 3′, 5′ to 5′ or 2′ to 2′ linkage. Modified backbones that do not include a phosphorus atom are formed by short chain alkyl or cycloalkyl internucleoside linkages, mixed heteroatom and alkyl or cycloalkyl internucleoside linkages, or one or more short chain heteroatomic or heterocyclic internucleoside linkages. Such backbones include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

In another example, the nucleic acid used in the disclosed methods includes one or more substituted sugar moieties. Such substituted sugar moieties can include one of the following groups at their 2′ position: OH; F; O-alkyl, S-alkyl, N-alkyl, O-alkenyl, S-alkenyl, N-alkenyl; O-alkynyl, S-alkynyl, N-alkynyl, and O-alkyl-O-alkyl. In these groups, the alkyl, alkenyl and alkynyl can be substituted or unsubstituted C₁ to C₁₀ alkyl or C₂ to C₁₀ alkenyl and alkynyl. They may also include at their 2′ position heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide. Preferred substituted sugar moieties include those having 2′-methoxyethoxy, 2′-dimethylaminooxyethoxy, and 2′-dimethylaminoethoxyethoxy. See Martin et al., Helv. Chim. Acta, 1995, 78, 486-504.

In yet another example, the nucleic acid includes one or more modified native nucleobases (i.e., adenine, guanine, thymine, cytosine and uracil). Modified nucleobases include those described in U.S. Pat. No. 3,687,808, The Concise Encyclopedia Of Polymer Science And Engineering, pages 858-859, Kroschwitz, J. I., ed. John Wiley & Sons, 1990, Englisch et al., Angewandte Chemie, International Edition, 1991, 30, 613, and Sanghvi, Y. S., Chapter 15, Antisense Research and Applications, pages 289-302, CRC Press, 1993. Certain of these nucleobases are particularly useful for increasing the binding affinity of the antisense oligonucleotide to its target nucleic acid. These include 5-substituted pyrimidines, 6-azapyrimidines and N-2, N-6 and O-6 substituted purines (e.g., 2-aminopropyl-adenine, 5-propynyluracil and 5-propynylcytosine). See Sanghvi, et al., eds., Antisense Research and Applications, CRC Press, Boca Raton, 1993, pp. 276-278).

Any of the nucleic acids can be synthesized by methods known in the art. See, e.g., Caruthers et al., 1992, Methods in Enzymology 211, 3-19, Wincott et al., 1995, Nucleic Acids Res. 23, 2677-2684, Wincott et al., 1997, Methods Mol. Bio. 74, 59, Brennan et al., 1998, Biotechnol Bioeng., 61, 33-45, and Brennan, U.S. Pat. No. 6,001,311. It can also be transcribed from an expression vector and isolated using standard techniques.

In other embodiments, the IL-20 antagonist comprises at least one IL-20 or IL-20R inhibitory compound. As used herein, “IL-20 inhibitory compound” or “IL-20R inhibitory compound” refers to a compound other than an anti-IL-20 or anti-IL-20R antibody that directly or indirectly reduces, inhibits, neutralizes, or abolishes IL-20/IL-20R biological activity. An IL-20/IL-20R inhibitory compound should exhibit any one or more of the following characteristics: (a) binds to IL-20 or IL-20R and inhibits its biological activity and/or downstream pathways mediated by IL-20 signaling function; (b) prevents, ameliorates, or treats any aspect of the target liver disease described herein; (c) blocks or decreases IL-20 receptor activation; (d) increases clearance of IL-20 or IL-20R; (e) inhibits (reduces) IL-20 or IL-20R synthesis, production or release. One skilled in the art can prepare other small molecules inhibitory compounds.

In other embodiments, the IL-20 or IL-20R inhibitory compounds described herein are small molecules, which can have a molecular weight of about any of 100 to 20,000 daltons, 500 to 15,000 daltons, or 1000 to 10,000 daltons. Libraries of small molecules are commercially available. The small molecules can be administered using any means known in the art, including inhalation, intraperitoneally, intravenously, intramuscularly, subcutaneously, intrathecally, intraventricularly, orally, enterally, parenterally, intranasally, or dermally. In general, when the IL-20-antagonist according to the invention is a small molecule, it will be administered at the rate of 0.1 to 300 mg/kg of the weight of the patient divided into one to three or more doses. For an adult patient of normal weight, doses ranging from 1 mg to 5 g per dose can be administered.

The above-mentioned small molecules can be obtained from compound libraries. The libraries can be spatially addressable parallel solid phase or solution phase libraries. See, e.g., Zuckermann et al. J. Med. Chem. 37, 2678-2685, 1994; and Lam Anticancer Drug Des. 12:145, 1997. Methods for the synthesis of compound libraries are well known in the art, e.g., DeWitt et al. PNAS USA 90:6909, 1993; Erb et al. PNAS USA 91:11422, 1994; Zuckermann et al. J. Med. Chem. 37:2678, 1994; Cho et al. Science 261:1303, 1993; Carrell et al. Angew Chem. Int. Ed. Engl. 33:2059, 1994; Carell et al. Angew Chem. Int. Ed. Engl. 33:2061, 1994; and Gallop et al. J. Med. Chem. 37:1233, 1994. Libraries of compounds may be presented in solution (e.g., Houghten Biotechniques 13:412-421, 1992), or on beads (Lam Nature 354:82-84, 1991), chips (Fodor Nature 364:555-556, 1993), bacteria (U.S. Pat. No. 5,223,409), spores (U.S. Pat. No. 5,223,409), plasmids (Cull et al. PNAS USA 89:1865-1869, 1992), or phages (Scott and Smith Science 249:386-390, 1990; Devlin Science 249:404-406, 1990; Cwirla et al. PNAS USA 87:6378-6382, 1990; Felici J. Mol. Biol. 222:301-310, 1991; and U.S. Pat. No. 5,223,409).

In other embodiments, the IL-20 antagonists can be a polypeptide comprising an extracellular portion of an IL-20 receptor (such as IL-20 R1, IL-20R2, or IL-22R1), wherein the polypeptide specifically binds to 11-20 and blocks its interaction with one or more IL-20 receptors. In some embodiments, the extracellular portion of the IL-20 receptor is fused to a Fc domain of antibody. Examples of the soluble receptors are described in PCT WO 01/46232.

(iv) Identification of IL-20 Antagonists

IL-20 antagonists can be identified or characterized using methods known in the art, whereby reduction, amelioration, or neutralization of an IL-20 biological activity is detected and/or measured. For example, an ELISA-type assay may be suitable for qualitative or quantitative measurement of IL-20 mediated kinase activation by measuring the phosphorylation of proteins activated through an IL-20 cascade. Examples include JNK, ERK, AKT, p38, STAT3 and TRAF6.

The IL-20 antagonists can also be identified by incubating a candidate agent with IL-20 or IL-20R and monitoring any one or more of the following characteristics: (a) binding to IL-20 or IL-20R and inhibiting its biological activity and/or downstream pathways mediated by IL-20 signaling function; (b) preventing, ameliorating, or treating any aspect of a liver disease such as those described herein; (c) blocking or decreasing IL-20 receptor activation; (d) increasing clearance of IL-20 or IL-20R; (e) inhibiting (reducing) IL-20 synthesis, production or release. In some embodiments, an IL-20 antagonist is identified by incubating a candidate agent with IL-20 or IL-20R and monitoring binding and attendant reduction or neutralization of a biological activity of IL-20 or IL-20R. The binding assay may be performed with purified IL-20 or IL-20R polypeptide(s), or with cells naturally expressing, or transfected to express, IL-20 or IL-20R polypeptide(s). In one embodiment, the binding assay is a competitive binding assay, where the ability of a candidate antibody to compete with a known IL-20 antagonist for IL-20 or IL-20R binding is evaluated. The assay may be performed in various formats, including the ELISA format. In other embodiments, an IL-20 antagonist is identified by incubating a candidate agent with IL-20 or IL-20R (e.g., IL-20R1) and monitoring attendant inhibition of IL-20R1/IL-20R2 complex formation or IL-20R2/IL-22R1 complex formation. Following initial identification, the activity of a candidate IL-20 antagonist can be further confirmed and refined by bioassays, known to test the targeted biological activities. Alternatively, bioassays can be used to screen candidates directly.

The examples provided below provide a number of assays that can be used to screen candidate IL-20 antagonists. Bioassays include but are not limited to flow cytometry of determine competitive binding of IL-20 to cells in the presence of candidate IL-20 antagonists; and inhibition of IL-20-induced apoptosis in renal epithelial cells. In addition, RT-PCR or Real-time PCR which can be used to directly measure IL-20 expression or to measure expression of genes upregulated by IL-20 such as TNFα MCP-1, IL-1β, IL-6 and VEGF.

### General Techniques

The practice of the present disclosure will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook, et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel, et al., eds., 1987); PCR: The Polymerase Chain Reaction, (Mullis, et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Example 1: Therapeutic Efficacy of IL-24 in Thioacetamide (TAA)-Induced Liver Disease Mouse Model

Periodic administration of thioacetamide (TAA) is the most commonly used approach to induce toxin mediated experimental liver fibrosis in animal models (e.g., in mice) for studying liver diseases such as liver fibrosis and evaluating treatment efficacy of drug candidates. TAA is bioactivated in the liver via oxidation processes, leading to the production of S-oxide and highly reactive S-dioxide, which is responsible for TAA hepatotoxity.

In this example, a TAA-induced liver fibrosis mouse model was established and the treatment efficacy of IL-24 was investigated. The results indicate that IL-24 increased the survival rate of the mice after TAA treatment to the level similar to that of healthy controls. IL-24 was also found to alleviate the body weight loss after the liver injuries. The H/E staining of liver sections from TAA-treated mice demonstrated that IL-24 reduced necrosis of the liver. Furthermore, the two indicators of liver function, ALT and AST, were also significantly inhibited by IL-24 treatment. The molecules of fibrosis and inflammation markers were all reduced after IL-24 treatment. These results demonstrated that IL-24 plays a pivotal role in recovery from liver injuries and thus it could be used as a therapeutic for liver fibrosis, and cirrhosis.

To investigate the protective effects of IL-24 on short-term TAA-induced liver injury, C57BL/6Jnarl mice were treated with PBS (control), mouse IL-24 (mIL-24) produced and purified from Escherichia coli cells, TAA or a combination of mIL-24 and TAA by intraperitoneal injection. IL-24 was given at 1 mg/kg either alone, or 24 hours before and 48 hours after the TAA treatment. The mice were sacrificed 72 hours after the treatment. Survival rates, body weight loss, H/E staining, and serum AST and ALT were determined by routine methodology at various time points as indicated.

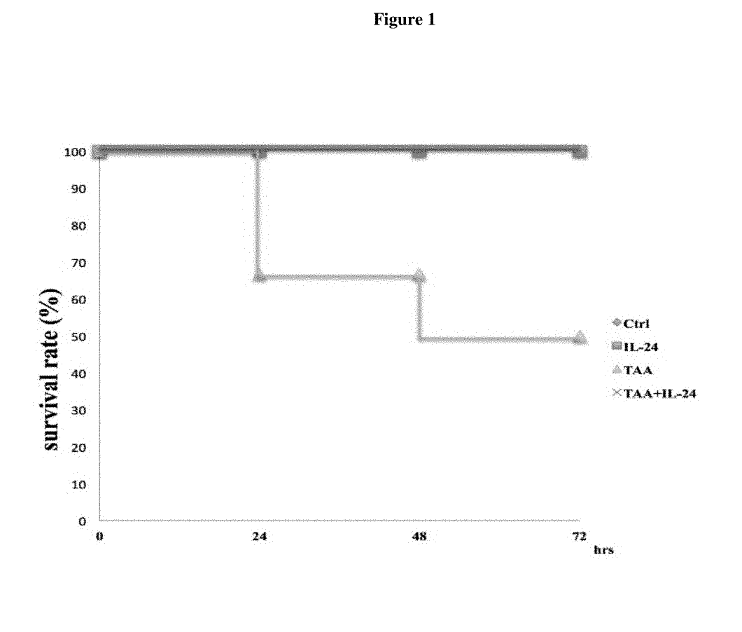

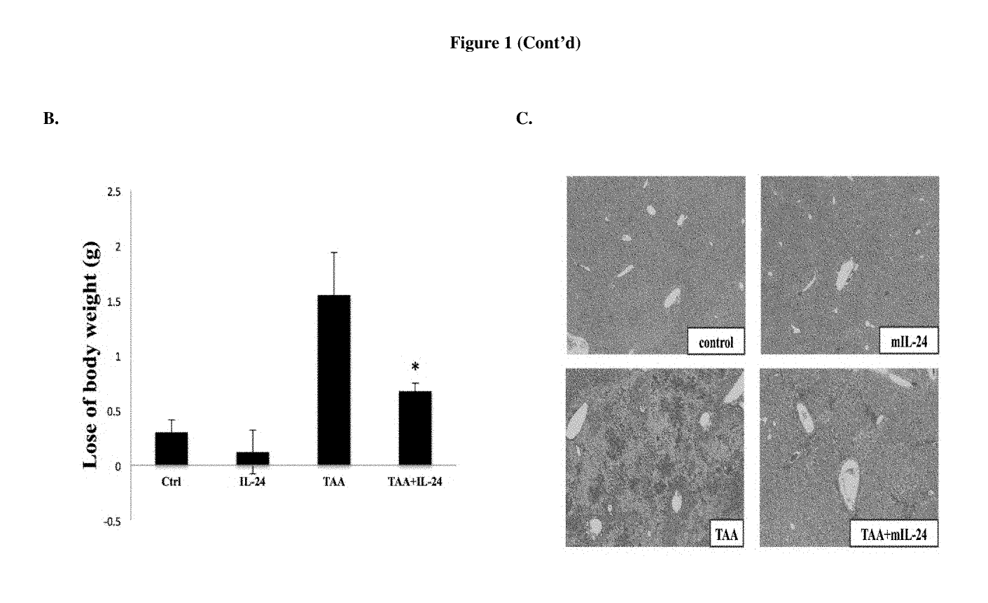

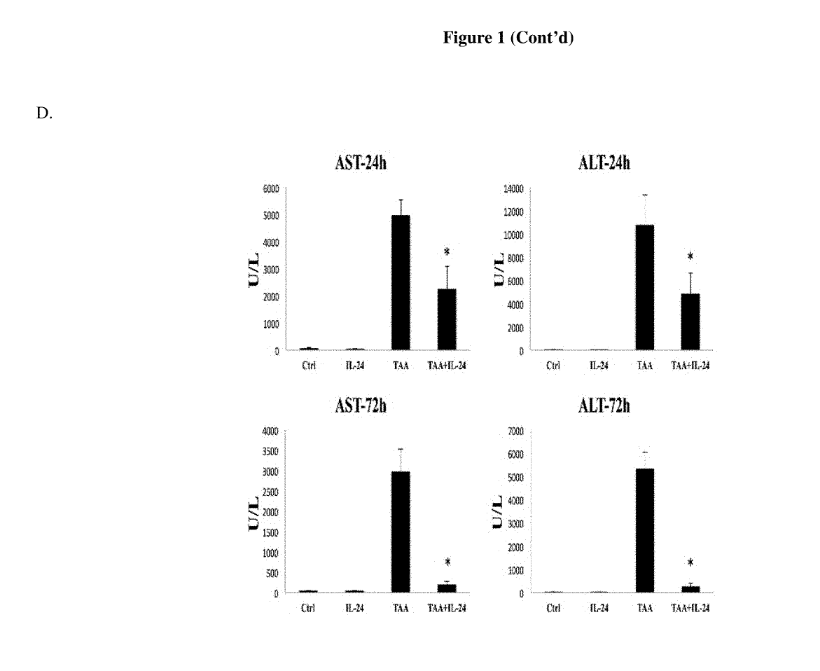

As shown in FIG. 1, panel A, mIL-24 treated mice showed the highest survival rate 72 hours after the treatment and the survival rate of IL-24 treated mice was substantially similar to that of the healthy control mice. Further, mIL-24 treated mice also showed less body weight loss as compared with the control mice. FIG. 1, panel B. Necrosis areas of livers were significantly decreased in mIL-24-treated mice as compared to mice treated with TAA alone (FIG. 1, panel C) and the serum levels of ALT and AST were also significantly lower in mIL-24-treated mice at 24 and 72 h after TAA treatment as compared with mice treated with TAA alone (FIG. 1, panel D).

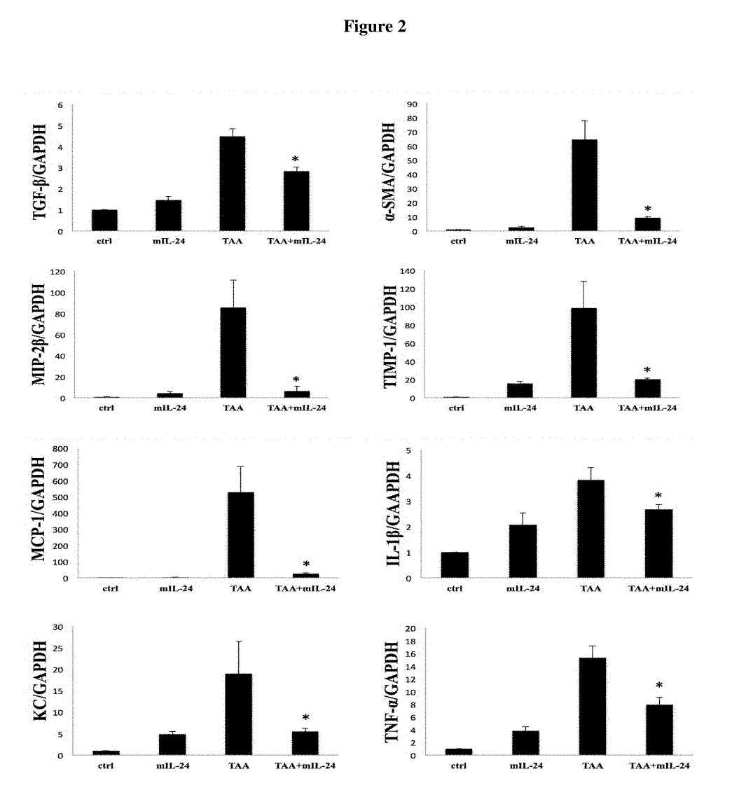

The levels of fibrosis markers (e.g., pro-fibrogenic cytokines) and inflammation markers (e.g., pro-inflammatory cytokines), such as TGF-β, α-SMA, MIP-2β, TIMP-1, MCP-1, IL-1β, KC and TNF-α, in the liver tissues obtained from the treated mice were examined by quantitative reverse transcription polymerase chain reaction (qRT-PCR) and the results are shown in FIG. 2. The levels of these fibrosis and inflammatory markers were significantly reduced in mIL-24-treated mice as compared with mice treated with TAA alone, indicating that IL-24 treatment is effective in reducing fibrosis and/or inflammation associated with liver injury.

Taken together, the data obtained from this study indicate that IL-24 plays a protective role in liver disease and could be used as a therapeutic drug for liver diseases.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS AND SCOPE

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A method for alleviating or delaying the onset of a liver disease, comprising administering to a subject in need thereof an effective amount of a pharmaceutical composition comprising an interleukin 24 (IL-24) protein.

2. The method of claim 1, wherein the liver disease is selected from the group consisting of liver fibrosis, liver cirrhosis, and liver steatosis.

3. The method of claim 1, wherein the subject is a human patient having or suspected of having the liver disease.

4. The method of claim 3, wherein the liver disease is liver fibrosis.

5. The method of claim 4, wherein the liver fibrosis is associated with chronic HBV infection, chronic HCV infection, alcohol abuse, nonalcoholic steatohepatitis, autoimmune hepatitis, primary biliary cirrhosis, fatty liver disease, liver cancer, or an idiopathic liver disease.

6. The method of claim 1, wherein the IL-24 protein is a human IL-24.

7. The method of claim 1, wherein the IL-24 protein is administered by a systemic route.

8. The method of claim 7, wherein the IL-24 protein is administered parenterally.

9. The method of claim 8, wherein the IL-24 protein is administered via intravenous infusion or intraperitoneal injection.

10. The method of claim 1, wherein the method further comprises administering to the subject an effective amount of a pharmaceutical composition comprising an interleukin 20 (IL-20) antagonist.

11. The method of claim 10, wherein the IL-20 antagonist is an antibody binding to human IL-20 or an antibody binding to a human IL-20 receptor.

12. The method of claim 11, wherein the antibody binds to subunit R1 of a human IL-20 receptor.

13. The method of claim 11, wherein the antibody is a full-length antibody or an antigen-binding fragment thereof.

14. The method of claim 11, wherein the antibody is a human antibody or a humanized antibody.
